(19) United States
(12) Patent Application Publication
(10) Pub. No.: US 2008/0118494 A1
(43) Pub. Date: May 22, 2008

(54) Compositions For Detection Of Latent Hiv Reactivation And Methods Of Using The Same

(76) Inventors: Olaf Kutsch, Birmingham, AL (US); Jennifer Jones, Vestavia Hills, AL (US); George M. Shaw, Birmingham, AL (US); David N. Levy, New York, NY (US)

Correspondence Address: NEEDLE & ROSENBERG, P.C., SUITE 1000, 999 PEACHTREE STREET, ATLANTA, GA, 30309-3915, US

(21) Appl. No.: 11/661,881

(22) PCT Filed: Sep. 2, 2005
(86) PCT No.: PCT/US05/31432

§ 371 Date: Nov. 8, 2007

(30) Foreign Application Priority Data
Sep. 2, 2004 (US) 60606561
Sep. 24, 2004 (EP) 10948753

(51) Int. Cl. A61K 35/76; C12N 7/00; C12N 5/06; C12Q 1/70; A61P 43/00; A61K 39/00; A61K 39/395

(52) U.S. Cl. 424/130.1; 435/235.1; 435/372.3; 435/5; 435/375; 424/278.1; 424/93.7

(57) ABSTRACT

Provided herein are compositions and methods that allow for the study of HIV latency and reactivation. Further provided are compositions and methods for in vitro screening of agents for their ability to reactivate, suppress reactivation or inhibit transcription of HIV. Compositions for and methods for activating a cell are also provided herein. Further provided herein are methods of treating a subject using agents that reactivate latent HIV infection or agents that inhibit HIV transcription. Also provided herein are methods of activating a latent microbiological entity in a subject. Further provided herein are methods for enhancing an immune response in a subject and compositions used as a vaccination adjuvant. Methods of making disclosed cells and compositions are also provided herein.

### CROSS REFERENCE TO RELATED APPLICATIONS

This application claims the benefit of U.S. Provisional Application No. 60/606,561, filed Sep. 2, 2004, which is hereby incorporated herein by reference in its entirety.

### BACKGROUND OF THE INVENTION

HIV-1 latency is a major obstacle to effective, lifelong control of HIV-1 infection and has been the nemesis of curative strategies for the disease. The failure of Highly Active Anti-Retroviral Therapy (HAART), the state of the art for HIV-1 infected patients, to eradicate the virus, has been mainly attributed to the inability of the currently used drugs to target the pool of latently HIV-1 infected cells. Successful HIV-1 therapy requires therapeutic reactivation of latent virus, thereby making the infected cells and virus vulnerable to immune clearance and drug treatment.

There was initial enthusiasm in strategies designed to “flush” cells out of latency. Interleukin-2 and antibodies to CD3 induce T-cell activation in vivo and conceivably could do this. However, studies in patients have shown significant toxicities from this therapy and no meaningful decay of the latent reservoir (Dybul et al., 2002). Recently, two groups have demonstrated that a naturally occurring, non-tumor-promoting phorbol ester, prostratin, induces the expression of latent HIV-1 in resting CD4⁺ T-cells without inducing cellular proliferation or enhancing de novo HIV-1 infection (Korin et al., 2002; Kulkosky et al., 2001), but the therapeutic index of prostratin will not allow for clinical use. An ideal HIV-1 reactivating agent thus needs to induce expression of latent HIV-1 by either directly targeting and activating the viral promoter or by activating the cellular reservoirs, while causing no or limited adverse side effects.

Investigators have studied HIV-1 latency in transformed cell lines such as ACH-2, J1.1, U1 and OM-10.1 (Bednarik, Cook, and Pitha, 1990; Brooks et al., 2001; Bushman and Craigie, 1992; Butera, 2000; Chun et al., 1999). These cell lines contain one or two copies of integrated virus but constitutively displayed low levels of HIV-1 gene expression. Moreover, their readouts for HIV-1 expression were relatively insensitive, generally p24 antigen production or viral RNA expression by in situ or northern blot analysis. Moreover, the state of latency in these cells, on a population basis or at the single cell level, could only be determined by indirect and time-consuming procedures (i.e., p24 ELISA, RT assay, intracellular staining for viral proteins).

As such, research on HIV-1 latency lacked a relevant model that was amenable to rapid and efficient analysis, and through which useful pharmacological compounds capable of effecting HIV-1 reactivation, could be efficiently screened.

Several reporter cell lines have been published in which enhanced green fluorescence protein (EGFP) serves a direct and quantitative marker of HIV-1 expression (Kutsch et al., 2002). These cell lines, for the first time, allowed analysis of HIV-1 reactivation at the single cell level and to correlate EGFP as an indicator of HIV-1 expression with other cellular markers (e.g. activation markers, apoptosis) using flow cytometric analysis. Several similar reporter cell lines (J-Lat Tat-GFP) (Jordan, Bisgrove, and Verdin, 2003) have also been published, emphasizing the attractiveness of this approach. Although these reporter cells proved extremely useful for flow cytometry based analysis, the cells are not optimal for plate-based fluorometry, a problem that can be explained by the, in comparison to flow cytometry, generally lower sensitivity of plate readers for fluorescent signals. The resulting relatively low dynamic range of the cell lines on plate based fluorometers renders the cells sub-optimal for high throughput screening (HTS). HTS using these cells would require expensive and time-consuming repeated screens and counter screens to assure the accuracy of the obtained data. HTS using the J-Lat Tat-GFP cells in a plate based 96-well or 384-well format is problematic, as the fluorescent signal upon reactivation has been relatively low.

In order to discover or develop drugs that therapeutically reactivate or activate latent human immunodeficiency virus, a system that allows rapid and high throughput screening for drugs with the capacity to reactivate or activate HIV infection is needed. Similar capabilities are needed to discover or develop drugs that inhibit HIV transcription.

### SUMMARY OF THE INVENTION

Provided herein are compositions and methods that allow for the study of HIV latency and reactivation. Further provided are compositions and methods for in vitro screening of agents for their ability to reactivate, suppress reactivation or inhibit transcription of HIV. Compositions for and methods for activating a cell are also provided herein.

Further provided herein are methods of treating a subject using agents that reactivate latent HIV infection or agents that inhibit HIV transcription. Also provided herein are methods of activating a latent microbiological entity in a subject. Further provided herein are methods for enhancing an immune response in a subject and compositions used as a vaccination adjuvant.

Methods of making disclosed cells and compositions are also provided herein.

Additional advantages will be set forth in part in the description which follows, and in part will be obvious from the description, or may be learned by practice of the aspects described below. The advantages described below will be realized and attained by means of the elements and combinations particularly pointed out in the appended claims. It is to be understood that both the forgoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows increased EGFP expression in LWI6 cells following reactivation of latent HIV-1 infection. JNLGFP and LWI6 cells were stimulated with TNF-α and after 48 h EGFP expression was determined by flow cytometry and compared to unstimulated JNLGFP (C).

FIGS. 2 (A and B) shows EGFP signal intensity and cell proliferation in relation to cell density. (A) JEGFP cells were seeded at the indicated cell density into individual wells of a 384-well plate and EGFP fluorescence was determined using a plate based fluorometer after 24 h. (B) JEGFP cells were seeded at the indicated cell densities and the increase of EGFP expression as a measure of cell proliferation was followed over a period of four days.

FIG. 3 shows a Z′-test for LWI6 cells. LWI6 cells were manually seeded at a cell density of 1×10⁵ cells/well in 384-well plates in a total volume of 80 μl phenol red free RPMI supplemented with 5% FBS. The 96 wells of the lower left and the upper right quadrant of each plate were then stimulated with TNF-α by manually adding 10 μl of a TNF-α solution that resulted in a final TNF-α concentration of 10 ng/ml. The wells in the upper left and lower right quadrant were treated with 10 μl of phenol red free RPMI. After 24 h, EGFP fluorescence was determined by plate based fluorometry. The graph depicts the results of three independent experiments.

FIG. 4 shows correlation of p24 expression, secretion of infectious viral particles and EGFP expression in LWI6 cells. LWI6 cells were stimulated with various concentrations of TNF-α or left unstimulated (C) for 48 h. At this timepoint, EGFP expression was determined by flow cytometric analysis (EGFP), expression of HIV-1 p24 Gag protein was determined by ELISA and the secretion of infectious viral particles (I.U.) was determined by titration of the supernatants on the JC53 indicator cell line.

FIG. 5 shows determination of compound influences on cell proliferation using JEGFP cells. JEGFP cells were seeded at a cell density of 1×10⁴ cells/well into a 384-well plate and left untreated (Control) or treated with a low concentration of daunorubicin (0.0001 μg/ml). EGFP fluorescence as a measure of cell proliferation was then determined over a period of four days.

FIGS. 6 (A, B and C), shows drug influence on the cytotoxic activity of CD8-positive T cells. To determine an influence of the test compounds on the cytotoxic activity of CD8-positive T cells, defined number of JLTRG/CUCY cells (target: T) were mixed at different ratios with PHA-L stimulated primary T cells that function as effector cells (effector: E). For the assay 2×10⁴ Calibrite beads, which based on their size and density can be readily distinguished from the cells (see R2 in B and C), were added to each culture, which was then subjected to flow cytometric analysis. The flow cytometer was adjusted to count 7500 beads, while acquiring all cells. The percentage of EGFP-positive JEGFP cells (lower right quadrant) was then used to determine the cytotoxic activity of the primary T cells (lower left quadrant). (A) Elimination of JEGFP cells as a function of an increasing effector to target ratio (E:T). (B) Flow cytometry based detection of JEGFP cells at a low E:T ratio of 0.1 after 48 h. (C) Flow cytometry based detection of JEGFP cells at a high E:T ratio of 30 after 48 h.

FIGS. 7 (A and B) shows quantitative assessment of HIV-1 latency formation. (A) Jurkat cells were infected with an EGFP expressing, replication incompetent reporter virus and the level of productive HIV-1 infection at each indicated time-point was directly assessed by flow cytometric analysis for EGFP expression (untreated; black circles). Levels of initial non-productive and later on latent HIV-1 infection was determined 24 hours following TNF-α stimulation (black squares). The results represent the mean ±S.D. of three independent experiments. (B) Activation of the bulk population of latently HIV-1 infected cells generated in (A; day 57) with the various HIV-1 reactivating agents indicated demonstrates the differences in potency of the agents to reactivate latent HIV-1 infection.

FIGS. 8 (A and B) shows HIV-1 latency and CD28-mediated HIV-1 reactivation in in vitro HIV-1 infected PBMCs. PBMCs from three different donors were infected with HIV-1 NLENG1-IRES and on day three post infection were treated with indinavir to inhibit de novo infection. On day seven following infection, HIV-1 expressing EGFP⁺ cells were removed from the cultures using fluorescence activated cell sorting. (A) Following the sorting procedure, the EGFP-negative cells were cultured in supplemented RPMI 1640 to determine levels of spontaneous reactivation (Control), stimulated with anti-CD3 antibody clone UCHT1 (CD3), the costimulatory anti-CD28 antibody clone CD28.2 (cCD28) and with a combination of these two antibodies (cCD28/CD3). These conditions were compared to the ability of activating anti-CD28 antibody clone 5D10 (aCD28) and a combination of this antibody and UCHT1 (aCD28/CD3) to reactivate latent HIV-1 infection. Numbers indicate the percentage of EGFP positive cells and the mean channel fluorescence intensity of the EGFP-positive cell population. (B) Reactivation of latent HIV-1 in PBMCs from three different donors by PMA, anti-CD3-, costimulatory anti-CD28-, activating anti-CD28 antibody and combinations thereof, presented as fold induction of EGFP expressing cells over background. Black bars represent the mean induction of three independent experiments.

FIGS. 9 (A, B and C) shows reactivation of latent HIV-1 infection by various T cell specific antibodies. J89GFP cells were stimulated with a single dose of various T cell specific antibodies (1 μg/ml) for 48 h and then subjected to flow cytometric analysis or fluorescence microscope analysis for EGFP fluorescence as a quantitative marker of HIV-1 expression. (A) J89GFP cells were left untreated, stimulated with anti-CD3 (clone HIT3a), anti-CD3 (clone UCHT1), activating anti-CD28 (clone 5D10), costimulatory anti-CD28 (clone L293), costimulatory anti-CD28 (clone CD28.2), and the following combinations of anti-CD3/anti-CD28 antibodies: 5D10/UCHT1, 5D10/HIT3a, CD28.2/UCHT1 and CD28.2/HIT3a. Anti-CD2 antibody stimulation (clone RPA-2.10) and TNF-α (10 ng/ml), served as negative and positive control, respectively. Numbers indicate the percentage of EGFP⁺ cells and the EGFP mean channel fluorescence intensity of the EGFP⁺ population. (B) Presentation of the data depicted in (A) as mean ±S.D. of four independent experiments. (C) Fluorescence and bright field microscopic view of unstimulated J89GFP cells (UN) and following stimulation with anti-CD3 antibody (UCHT1), activating anti-CD28 antibody (5D10) and simultaneous stimulation with an anti-CD3- and a costimulatory anti-CD28 antibody (UCHT1/CD28.2). The results are representative for four independent experiments.

FIG. 10 (A, B and C) shows specificity and potency of activating anti-CD28 antibody mediated HIV-1 reactivation. (A) Accumulative effect of activating anti-CD28 antibody on HIV-1 reactivation in J89GFP cells following three repeated additions (3×5D10), compared to unstimulated J89GFP cells (UN) and J89GFP cells following a single dose treatment with 5D10, and inhibition of HIV-1 reactivation mediated by activating anti-CD28 antibody (clone 5D10) by preincubation with two different costimulatory anti-CD28 antibodies (CD28.2/5D10 and L293/5D10). The results are representative for five independent experiments. (B) CD28 expression on J89GFP cells detected using anti-CD28 antibodies clone 5D10, clone L293 and clone CD28.2. (C) J89GFP cells were pretreated for two hours with the specific ERK-inhibitor U0126 (control: U0124) or the specific p38-inhibitor SB202190 (control: SB202474) and then stimulated with activating anti-CD28 antibody 5D10 (1 μg/ml). Level of reactivation was measured as %-EGFP⁺ cells using flow cytometry. Results represent the mean ±S.D. of three independent experiments.

FIG. 11 (A and B) shows HIV-1 latency and CD28-mediated HIV-1 reactivation in in vitro HIV-1 infected PBMCs. PBMCs from three different donors were infected with HIV-1 NLENG1-IRES and on day three post infection were treated with indinavir to inhibit de novo infection. On day seven following infection, HIV-1 expressing EGFP⁺ cells were removed from the cultures using fluorescence activated cell sorting. (A) Following the sorting procedure, the EGFP-negative cells were cultured in supplemented RPMI 1640 to determine levels of spontaneous reactivation (Control), stimulated with anti-CD3 antibody clone UCHT1 (CD3), the costimulatory anti-CD28 antibody clone CD28.2 (cCD28) and with a combination of these two antibodies (cCD28/CD3). These conditions were compared to the ability of activating anti-CD28 antibody clone 5D10 (aCD28) and a combination of this antibody and UCHT1 (aCD28/CD3) to reactivate latent HIV-1 infection. Numbers indicate the percentage of EGFP positive cells and the mean channel fluorescence intensity of the EGFP-positive cell population. (B) Reactivation of latent HIV-1 in PBMCs from three different donors by PMA, anti-CD3-, costimulatory anti-CD28-, activating anti-CD28 antibody and combinations thereof, presented as fold induction of EGFP expressing cells over background. Black bars represent the mean induction of three independent experiments.

FIG. 12 (A and B) shows susceptibility of PBMCs to HIV-1 infection following stimulation with various T cell specific antibodies and antibody combinations. PBMCs from seven donors were stimulated for 48 h with the indicated T cell specific antibodies and antibody combinations at a concentration of 1 μg/ml for each antibody. The following antibodies were used: (anti-CD2 RPA-2.10); anti-CD3 (UCHT1); anti-CD3 (HIT3a); activating anti-CD28 (5D10); costimulatory anti-CD28 (CD28.2); costimulatory anti-CD28 (L293). The cells were then infected with NLENG1-IRES (MOI: 0.01). Five days post infection the cells were subjected to flow cytometric analysis to determine (A) levels of infection (% EGFP expression) and (B) the induction of cell proliferation.

FIG. 13 shows CD28-mediated HIV-1 reactivation in latently infected thymocytes derived from HIV-1 infected SCID-hu (Thy/liv) Mice. SCID-hu (Thy/liv) mice were infected with HIV-1 NLENY1-IRES, and thymocytes were isolated 2-4 weeks following infection. Cells were cultured for two days in the presence of AZT and indinavir to stop de novo infection and then subjected to fluorescence activated cell sorting to remove all EYFP⁺ cells. Immediately following cell separation, the cells were seeded at a concentration of 1×10⁶ cell/ml, cultured in supplemented RPMI 1640 to determine levels of spontaneous reactivation, stimulated with PMA, TNF-α, activating anti-CD28 antibody (clone 5D10) and the histone acetylase inhibitor sodium butyrate (NaBu; two animals only). Levels of reactivation achieved in HIV-1 infected thymocytes derived from 4 different SCID-hu (Thy/liv) mice following ex vivo reactivation with the indicated stimulators are depicted as fold induction of EGFP expression.

FIG. 14 shows determination of the HIV-1 suppressive potency of potential HIV-1 Tat inhibitors in activated cells. EGFP expression following TNF-α (1 ng/ml) mediated reactivation of latent HIV-1 infection in J89GFP cells in the presence or absence of Ro24-7429 (10 μM). Following stimulation, area (A+B) represents the total amount of virus produced over a two day period of time, whereas area B represents the amount of virus produced in the presence of Ro24-7429. Results represent the mean ±S.D. of three independent experiments.

FIG. 15 shows onset kinetics of Ro24-7429 mediated HIV-1 inhibition using LWI6 cells. EGFP expression following TNF-α (1 ng/ml) mediated reactivation of latent HIV-1 infection in LWI6 cells in the presence or absence of Ro24-7429 (10 μM). Following stimulation, area (A+B) represents the total amount of virus produced over a two day period of time, whereas area B represents the amount of virus produced in the presence of Ro24-7429. Results represent the mean ±S.D. of three independent experiments.

FIG. 16 shows a comparison of the level of HIV-1 reactivation induced in LWI6 and LWI6-R cells by various established HIV-1 reactivating agents.

FIG. 17 A shows the correlation of levels of HIV-1 reactivation as determined by flow cytometric analysis (FACS) and plate-based fluorometry in LWI6-R cells for EGFP expression. FIG. 17 B shows the correlation of levels of cell viability as determined by flow cytometric analysis (FACS) and plate-based fluorometry in LWI6-R cells for DSRedExpression.

### DETAILED DESCRIPTION

Before the present compounds, compositions, articles, devices, and/or methods are disclosed and described, it is to be understood that the aspects described below are not limited to specific synthetic methods or specific administration methods, as such may, of course, vary. It is also to be understood that the terminology used herein is for the purpose of describing particular aspects only and is not intended to be limiting.

It must be noted that, as used in the specification and the appended claims, the singular forms “a,” “an” and “the” include plural referents unless the context clearly dictates otherwise. Thus, for example, reference to “an agent” includes mixtures of agents, reference to “a pharmaceutical carrier” or “adjuvant” includes mixtures of two or more such carriers or adjuvants, and the like.

As used throughout, by a “subject” is meant an individual. Thus, the “subject” can include domesticated animals, such as cats, dogs, etc., livestock (e.g., cattle, horses, pigs, sheep, goats, etc.), laboratory animals (e.g., mouse, rabbit, rat, guinea pig, etc.) and birds. In one aspect, the subject is a mammal such as a primate or a human.

“Optional” or “optionally” means that the subsequently described event or circumstance can or cannot occur, and that the description includes instances where the event or circumstance occurs and instances where it does not. For example, the phrase “optionally the detectable reporter marker is a fluorescent protein” means that the detectable reporter marker may comprise a fluorescent protein or may not comprise a fluorescent protein such that the description includes both the fluorescent protein and the absence of the florescent protein.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Provided herein are compositions and methods that allow for the study of HIV latency and reactivation. Further provided are compositions and methods for in vitro screening of agents for their ability to reactivate, suppress reactivation or inhibit transcription of HIV. Compositions for and methods for activating a cell are also provided herein.

Provided herein is a cell comprising a stably integrated reporter plasmid. The reporter plasmid comprises a nucleic acid encoding a detectable reporter marker that is operatively linked to an immunodeficiency viral promoter. The cell also comprises a replication-competent or non-replication competent immunodeficiency virus integrated into the genome of the cell. Under basal in vitro culture conditions, the immunodeficiency virus is latent and the expression of the latent immunodeficiency virus can be activated. The cell can be used for in vitro screening of agents for their ability to reactivate, suppress reactivation or inhibit transcription of HIV. The cell can also be used to identify or to screen for a vaccine, or to identify agents or compositions for activating a cell, activating a latent pathogen, enhancing an immune response, or for treating a subject with an HIV or another infection. Optionally, the cell further comprises a stably integrated indicator plasmid. The indicator plasmid comprises a nucleic acid encoding a detectable indicator marker that is distinguishable from the detectable reporter marker. The detectable indicator marker is constitutively expressed under basal in vitro culture conditions. Further provided herein is a kit comprising a vessel or vessels containing the cell or a population of the cells.

The cell comprising the optional indicator plasmid allows for the simultaneous determination of a target agent's effect on latent HIV and on cell viability, which can greatly reduce the cost of screening. The cell comprising the optional indicator plasmid further reduces the likelihood of generating false positive hits (compounds that trigger HIV-1 reactivation due to compound cytotoxicity, as apoptosis can reactivate the latent virus) or of generating false negative hits (compounds that are toxic at the tested concentration, but are effective at decreased concentration levels). Thus, the cell comprising the optional indicator plasmid can be used to decrease the overall drug screening effort, as no additional cytotoxicity screen needs to be performed.

The term “latent,” as used herein in the context of a latent immunodeficiency virus refers to a genomically integrated immunodeficiency virus (including a latent immunodeficiency virus-based retroviral vector, e.g., a recombinant immunodeficiency virus) that is transcriptionally silent or inactive, e.g., immunodeficiency virus transcripts are undetectable or are at background levels, in a cell comprising the latent immunodeficiency virus.

As used herein “activation” and “activated” mean the same as “reactivation” and “reactivated” respectively. Therefore, as would be clear to one skilled in the art, these terms are used interchangeably herein. For example, the disclosure of a latent immunodeficiency virus that can be activated also discloses a latent immunodeficiency virus that can be reactivated as these terms both convey to the skilled artesian an state of transcriptional activity of an immunodeficiency virus as opposed to a state of latency characterized by transcriptional inactivity or silence.

The term “reactivated” or “activated,” as used herein in the context of in vivo reactivated immunodeficiency virus, refers to an immunodeficiency virus that, after a period of latency, becomes transcriptionally active, and in many instances forms infectious viral particles. The term “reactivated” or “activated”, as used herein in the context of in vitro reactivated immunodeficiency virus in a cell, refers to an immunodeficiency virus that, after a period of latency, becomes transcriptionally active, i.e., a functional Tat protein mediates transcription from a functional immunodeficiency virus promoter (e.g., a long terminal repeat promoter).

The latent immunodeficiency virus, in one example, HIV, is transcriptionally inactive under basal in vitro culture conditions but is fully replication competent. “Replication-competent” as used herein refers to an immunodeficiency virus that is capable of viral replication. “Non-replication competent” as used herein refers to an immunodeficiency virus that is incapable of viral replication due to random mutation or targeted changes in the viral genome that did not alter the viral promoter of the viral Tat-gene. For example, the latent immunodeficiency virus, in one example, HIV, is integrated in the cellular genome but is transcriptionally inactive under basal in vitro conditions and is “non-replication competent” due to a mutation in the gag-gene, the pol-gene, the vif-gene, the vpu-gene, the vpr-gene, the nef-gene, or the env-gene, which would not influence its ability to express EGFP as a direct and quantitative indicator of HIV-1 LTR activity. As used herein, an immunodeficiency virus integrated into the genome of a cell can be replication competent or non-replication competent. Thus, the terms replication competent and non-replication competent are used interchangeably herein.

The term “constitutively expressed” as used herein in the context of the expression of an optional detectable indicator marker means that, under basal in vitro culture conditions, the gene or genes encoding the indicator maker are expressed without additional stimulation. For example, a gene encoding DsRed-Express can be constitutively expressed such that the fluorescent protein encoded by the gene, DsRed-Express, is expressed in the cell under basal in vitro culture conditions. The optional detectable indicator marker is not, however, limited to DsRed-Express and can be selected from any detectable markers, so long as the detectable indicator marker is distinguishable from the detectable reporter marker. If a fluorescent detectable reporter marker is used, as described below, any florescent protein that is spectrally distinct from the detectable reporter marker can be used for the detectable indicator marker. Moreover, the detectable indicator marker is not limited to a fluorescent protein, and can be any detectable protein that is constitutively expressed in the cell. For example, the detectable protein can be selected from luciferase, soluble alkaline phosphatase, and a cell surface-expressed marker protein. The expression of the detectable indicator marker is similar to the expression of housekeeping genes in that the expression of the gene or genes encoding the detectable indicator marker is not altered by stimulation of the cell.

If the detectable indicator marker is a fluorescent protein, it can be detected using florescent detection techniques, which are known in the art and are described in greater detail below. Other methods for detecting expressed proteins can also be used to detect the indicator marker. For example, antibody staining and/or enzymatic detection techniques, or any other detection technique know in the art for detecting expressed proteins can be used to detect the detectable indicator marker and/or the detectable reporter marker.

“Basal in vitro culture conditions” typically involve standard culture media, a temperature of about 37° C., and 5% CO₂. Standard culture media can include, but are not limited to, RPMI 1640 medium, McCoy's 5A medium, Leibovitz's L15 medium, Eagle's minimal essential medium, Dulbecco's modified Eagle's medium, and the like. Optionally, the medium can be supplemented with additional components, e.g., 10 mM HEPES buffer; 2 mM L-glutamine; 100 U/ml penicillin; 100 μg/ml streptomycin; and heat-inactivated fetal bovine serum, in an amount (in volume/volume) of from about 2% to about 5% from about 5% to about 10%, from about 10% to about 15%, or from about 15% to about 20%, or higher. For example, 10% heat-inactivated fetal bovine serum can be used.

Basal in vitro culture conditions generally exclude the presence in the medium of a factor(s) that would activate HIV transcription and/or production of HIV virions, including the factors disclosed for reactivation of latent HIV. Under certain cell culture conditions, the latent HIV can be reactivated, e.g., the latent HIV becomes transcriptionally activated. Basal in vitro conditions also exclude the presence of an agent to be tested for its ability to activate HIV, inhibit HIV transcription, suppress HIV activation, or inhibit activation of a target cell.

Any of a variety of cells can comprise a replication-competent immunodeficiency virus or non-replication competent immunodeficiency virus integrated into the genome of the cell, a stably integrated reporter plasmid, and an optional integrated indicator plasmid. Optionally, the cell is an immortalized cell line. The cell optionally can be a primary cell culture and is not immortalized. Optionally, the cell is a T cell or a T cell line. For example, the cell can be a T cell or an immortalized T cell line that is permissive for an immunodeficiency virus, e.g., can be infected by an immunodeficiency virus, e.g., the T cell or immortalized T cell line expresses on its cell surface a CD4 receptor and a co-receptor (e.g., CXCR4 or CCR5). For example, the T-cell can be from the Jurkat lineage.

Suitable immortalized T cell lines include, but are not limited to, Jurkat; MOLT-16; MOLT-17; MOLT-3; MOLT-4, Karpas-299; HuT78; HSB-2; CCRF-CEM; SupT1; H9; and the like. Such cell lines are publicly available, e.g., from the American Type Culture Collection.

Primary cultures of T cells can be obtained using standard methods. For example, human peripheral blood mononuclear cells (PBMC) are removed from a human donor, and the T lymphocytes are separated from other lymphoid cells by any known method, including, but not limited to Ficoll-Hypaque cell separation. The cells can then be further subjected to cell sorting on the basis of cell surface expression of CD4 and CD3 molecules, e.g., using a fluorescence activated cell sorter and labeled antibody specific for CD4 and for CD3. The cells are then stimulated in the presence of PHA and grown continuously in the presence of low concentrations of recombinant IL-2, according to standard protocols.

Optionally, a disclosed cell is a clonal cell line. A cell can also be a member of a homogeneous population of cells (e.g., a population of cloned cells from a single cloned cell line). The immunodeficiency virus need not be integrated at the same genomic site in each cell of a population, and to that extent, the population can be considered heterogeneous, even though the cells used to make the population are from a single cell line.

The immunodeficiency viral promoter can be for example a promoter of HIV-1 or HIV-2. The immunodeficiency viral promoter can also be for example a promoter of SIV. Optionally, the immunodeficiency viral promoter is a human immunodeficiency virus, HIV-1, promoter. For example, the promoter can be the HIV-1 promoter, LTR, or long terminal repeat sequence.

The term “immunodeficiency virus” as used herein, refers to human immunodeficiency virus-1 (HIV-1); human immunodeficiency virus-2 (HIV-2); any of a variety of HIV subtypes and quasispecies; simian immunodeficiency virus (SIV); and feline immunodeficiency virus (FIV). Optionally, the latent replication competent or non-replication competent immunodeficiency virus can be human immunodeficiency virus (HIV). For example, the immunodeficiency virus can be HIV-1 or HIV-2.

The latent immunodeficiency virus can be wild-type or recombinant. Optionally, the latent immunodeficiency virus is recombinantly generated using standard recombinant DNA methods. Optionally, the latent replication competent or non-replication competent immunodeficiency virus is a wild-type immunodeficiency virus. HIV genome sequences are known in the art for a variety of HIV-1 and HIV-2 strains, and can be found in GenBank under various accession numbers, including AJ203647, AAAJ302646; AF133821, NC001802, L36874, and NC001722. SIV genome sequences are known in the art for a variety of SIV strains, and can be found in GenBank under various accession numbers, including AF334679, and NC001549. Any of a variety of strains and quasispecies can be used. Optionally, the HIV-1 can be from a primary patient isolate. For example, the primary patient isolate HIV-1 WI70, HIV-1 REJO or HIV-1 WEAU can be used.

The reporter plasmid comprises a nucleic acid encoding a detectable reporter marker. Optionally, the detectable reporter marker can be a protein. Suitable detectable reporter marker proteins include, but are not limited to, fluorescent proteins (e.g., a green fluorescent protein (GFP) (including enhanced GFP, e.g., available from Clontech); a fluorescent protein from an Anthozoa species (as described in, e.g., Matz et al. (1999) Nat. Biotech. 17:969-973); β-galactosidase; luciferase; and the like. For example, the detectable reporter marker can be enhanced green fluorescence protein (EGFP). As described above, the detectable indicator marker can also be a protein, including but not limited to, a fluorescent protein. If the detectable reporter marker used is a fluorescent protein having a typical spectrum of fluorescent emission, then the detectable indicator can be a fluorescent protein having a distinct spectral emission from that of the reporter marker. For example, if the detectable reporter marker is enhanced green fluorescent protein (EGFP) then the detectable indicator marker can be a fluorescent protein spectrally distinct form EGFP. One such spectrally distinct protein is DsRedExpress. Many combinations of spectrally distinct detectable reporter marker proteins and detectable indicator proteins can be used. Detection of the detectable reporter or detectable indicator marker can be carried out using a method suitable to the particular marker. For example, where the marker is a fluorescent protein, fluorescence is detected; where the marker is a luminescent protein, luminescence is detected.

The nucleotide sequence encoding the detectable reporter marker is operably linked to a promoter. Optionally, the promoter is an immunodeficiency virus promoter.

Provided herein are methods of identifying agents that activate or inhibit activation of latent immunodeficiency virus. Provided herein are methods of identifying an agent that activates a latent immunodeficiency virus, the method comprising, contacting the above disclosed cell with a test agent, detecting the detectable reporter marker, the detectable reporter marker indicating that the test agent activates the latent immunodeficiency virus.

Further provided herein are methods of identifying an agent that inhibits immunodeficiency virus transcription, comprising contacting the cell with a test agent, contacting the cell with an immunodeficiency virus activating agent and detecting the presence of detectable reporter marker, the level of which, when decreased as compared to a control level, indicating an agent that inhibits viral transcription.

Further provided herein is a method of identifying an agent that causes activation of a target cell, the method comprising contacting the cell with a test agent, detecting the detectable reporter marker, the detectable reporter marker indicating that the test agent activates the target cell. Optionally, the target cell can be a T-cell.

If the cell used in the disclosed methods comprises the optional indicator plasmid, the disclosed methods can further comprise detecting the detectable indicator marker. A stable level of the indicator marker as compared to a control level indicates that the test agent is not cytotoxic to the cell. A decrease in the level of the detectable indicator marker as compared to a control level indicates cytotoxicity of the test agent.

The terms “candidate agent,” “agent,” “substance,” and “test agent,” are used interchangeably herein. Candidate agents encompass numerous chemical classes, and are generally synthetic, semi-synthetic, or naturally occurring inorganic or organic molecules. Candidate agents may be small organic compounds having a molecular weight of more than 50 and less than about 2,500 daltons. Candidate agents, however, are not limited to compounds of this size. Candidate agents can also include peptides, polypeptides, antibodies, proteins (e.g., recombinant proteins), macromolecules and siRNA. Candidate agents can also include therapeutic viral vectors, for example adenoviral vectors as would be known to those skilled in the art. Candidate agents may comprise functional groups necessary for structural interaction with proteins, particularly hydrogen bonding, and may include at least an amine, carbonyl, hydroxyl or carboxyl group, and may contain at least two of the functional chemical groups. The candidate agents may comprise cyclical carbon or heterocyclic structures and/or aromatic or polyaromatic structures substituted with one or more of the above functional groups. Candidate agents are also found among biomolecules including glycoproteins, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

An agent can be assessed for any cytotoxic activity it may exhibit toward control cells not infected with an immunodeficiency virus, using well-known assays, such as trypan blue dye exclusion, an MTT ([3-(4,5-dimethylthiazol-2-yl)-2,5-diphenyl-2H-tetrazolium bromide]) assay, and the like.

The disclosed methods can include one or more controls. Thus, a control sample can have all the components of the test sample or agent except for the test agent.

Effective amounts of exemplary reactivating agents are as follows: from about 5 nM to about 10 nM TPA; from about 0.1 ng/ml to about 10 ng/ml TNF-α; from about 0.3 μg/ml to about 10 μg/ml anti-CD3 antibody; from about 30 nM to about 1 μM TSA; PMA/TPA at from about 0.3 ng/ml to about 30 ng/ml; Activating anti-CD28 antibody at from about 0.3 μg/ml to about 10 μg/ml; Soluble recombinant CD154 at from about 0.1 μg/ml to about 10 μg/ml; Sodium butyrate at from about 0.1 mM to about 1 mM; and Prostratin at from about 0.003 μM to about 3 μM. Non-limiting examples of effective amounts of exemplary reactivating agents are as follows: 10 nM TPA; 10 ng/ml TNF-α; 5 μg/ml anti-CD3 antibody; and 400 nM TSA. Other non-limiting examples of activating agents include PMA, Prostratin, IL-2, Histone deacetylase inhibitors (trichostatin A, sodium butyrate), Recombinant soluable CD 154, activating or superantagonistic anti-CD28 antibody, and agents identified using the methods described herein.

Suitable periods of time for contacting a cell with a reactivating agent are from about 0.5 hour to about 24 hours, e.g., from about 1 hour to about 2 hours, from about 2 hours to about 4 hours, from about 4 hours to about 8 hours, from about 8 hours to about 12 hours, from about 12 hours to about 16 hours, from about 16 hours to about 20 hours, or from about 20 hours to about 24 hours. Contacting a cell with an effective amount of a reactivating agent is typically conducted under standard culture conditions of 37° C. and 5% CO₂.

A variety of reagents can be included in the disclosed methods. For example, these include reagents like salts, neutral proteins, e.g. albumin, detergents, etc that can be used to facilitate optimal protein-protein binding and/or reduce non-specific or background interactions. Reagents that improve the efficiency of the assay, such as nuclease inhibitors, anti-microbial agents, also can be used. The components can be added in any order. Incubations can be performed at any suitable temperature, typically between 37° C. and 40° C. Incubation periods can be selected for optimum activity, but can also be optimized to facilitate rapid high-throughput screening.

Optionally, the detectable reporter marker and/or detectable indicator marker are fluorescent proteins, and detection of the markers is by flow cytometry, using a fluorescence activated cell sorter (FACS). Detection of the markers can also be by plate based fluorometry. For example, a single well plate based format, a 96 well plate based format, a 384 well plate based format, or any other well plate based format can be used. In 384 well plates from approximately 10,000 to approximately 200,000 cells per well in a total volume of 80-100 μl can be used. Detection of the markers can also be made by flow cytometry, or fluorometry, or fluorescence microscopy (automated) on live cells or on fixed cells.

For fluorometric analysis, EGFP requires no cofactors, stains, or other agents for detection besides a light source. Ideal excitation for EGFP, as given by the manufacturer (Clontech, Palo Alto, Calif.), is at 488 nm, and ideal emission is at 508 nm. As would be clear to one skilled in the art, however, other wave lengths of light can be used. For example, excitation at 435 nm and emission at 530 nm, or other suitable parameters can be used. For DsRed-Express, ideal excitation and emission spectra are 557 nm and 579 nm respectively.

Flow-cytometric analysis can be performed with a FACStar Plus, a FACScan, a LSR, an ARIA and CellQuest software (BD Biosciences, San Jose, Calif.), or with equivalent hardware and software.

A disclosed cell can also be photographed in culture using a Nikon TE300 inverted microscope and Hoffman optics (Modulation Contast, Inc., Greenvale, N.Y.) at ×100 by using a SenSys:140E B&W cooled charge-coupled device camera (Photometrics, Inc., Tucson, Ariz.), or another equivalent system. Optionally, to detect EGFP fluorescence in photomicroscopy, a Piston green fluorescent protein set can be used (Chroma, Inc., Rockingham, Vt.).

When a fluorescent reporter marker and/or indicator marker is used, the plates holding cells can be screened at the beginning of the cell culture (0 h) to account for the influence of compound autofluorescence. At this time, detectable fluorescence in the wavelength spectrum of the reporter marker is attributable to compound autofluorescence. Autofluorescence in the wavelength spectrum of the indicator marker is indicated by an increase in the level of the indicator marker fluorescence spectrum intensity that is characteristic for the constitutive expression of the indicator marker in the cells.

Further provided herein is a composition comprising an agent identified by the disclosed methods of identifying an agent that activates a latent immunodeficiency virus and a pharmaceutically acceptable carrier. Also provided herein is a composition comprising an agent identified by the disclosed method of identifying an agent that causes activation of a target cell, and a pharmaceutically acceptable carrier. Also provided herein is a composition comprising an agent identified by the disclosed method of identifying an agent that inhibits activation of a latent immunodeficiency virus, and a pharmaceutically acceptable carrier.

Further provided herein is a method of enhancing an immune response in a subject comprising administering to the subject an effective amount of an agent identified as causing activation of a target cell. Methods of assessing immune responses are well known in the art. The composition identified as causing activation of a target cell can be used as a vaccination adjuvant. An adjuvant can be a part of the carrier of the vaccine, in which case it can be selected by standard criteria based on the antigen used, the mode of administration and the subject (Arnon, R. (Ed.), 1987). Methods of administration can be by oral or sublingual means, or by injection, depending on the particular vaccine used and the subject to whom it is administered.

Also provided herein is a method of treating a subject infected with HIV comprising extracting a bone marrow stem cell population, peripheral blood stem cell population, or both from the subject, eradicating all T cells in the subject, contacting the extracted cell population with an effective amount of an agent identified using the disclosed methods of identifying agents that activate latent immunodeficiency virus, wherein contacting the extracted cell population with the agent activates latent HIV expression in the extracted latently infected cells, contacting the extracted cells with an effective amount of one or more agents that kills cells with active HIV expression, and transplanting the surviving non-HIV infected stem cells into the subject. Extracting cells from the subject's bone marrow or obtaining peripheral blood stem cells can be accomplished using common techniques known to those of skill in the art. T cells in the subject can be eradicated using techniques and protocols known in the art. Techniques and protocols for eradication of T cells in the subject include administration of chemotherapy and/or radiation to the subject. To kill extracted cells that are actively expressing HIV, an HIV-1 specific immunotoxin, e.g., can be used. HIV specific immunotoxins are known to those skilled in the art and can specifically target cells that are actively expressing HIV-1 or HIV-2. The immunotoxins can be used alone or in conjunction with one or more agents that inhibits a human immunodeficiency viral function, including those disclosed herein. As used herein, the terms “cell population,” “bone marrow cell population,” and “peripheral blood stem cell population,” refer to a population of cells that can comprise bone marrow stem cells, peripheral blood stem cells, as well as other cell types such as CD4+ cells that can be infected with immunodeficiency virus, and combinations thereof.

As disclosed above, the compositions can be administered in vivo in a pharmaceutically acceptable carrier. By “pharmaceutically acceptable” is meant a material that is not biologically or otherwise undesirable, i.e., the material may be administered to a subject, along with the agent identified by the disclosed methods, without causing any undesirable biological effects or interacting in a deleterious manner with any of the other components of the pharmaceutical composition in which it is contained. The carrier can be selected to minimize any degradation of the active ingredient and to minimize any adverse side effects in the subject, as would be well known to one of skill in the art.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. It will be apparent to those persons skilled in the art that certain carriers may be more preferable depending upon, for instance, the route of administration and concentration of composition being administered. Pharmaceutical carriers are known to those skilled in the art. These most typically would be standard carriers for administration of drugs to humans, including solutions such as sterile water, saline, and buffered solutions at physiological pH.

Provided herein is a method of activating a latent human immunodeficiency virus (HIV) in a cell comprising contacting the cell with an effective amount of the composition or agent of the composition identified by the disclosed method of identifying an agent that activates a latent immunodeficiency virus. Further provided herein is a method of inhibiting transcription in a cell comprising contacting the cell with an agent identified by the described methods of identifying an agent that inhibits immunodeficiency virus transcription.

Provided herein is a method of treating a subject with human immunodeficiency virus infection comprising administering to the subject an effective amount of the composition identified to activate latent immunodeficiency virus, and administering to the subject an effective amount of one or more agents that inhibits a human immunodeficiency viral function. For example, the agent or agents that inhibit the human immunodeficiency virus function are selected from the group consisting of a viral replication inhibitor, a viral protease inhibitor, a viral reverse transcriptase inhibitor, a viral entry inhibitor, a viral integrase inhibitor, a viral Rev inhibitor, a viral Tat inhibitor, a viral Nef inhibitor, a viral Vpr inhibitor, a viral Vpu inhibitor, and a viral Vif inhibitor.

Further provided herein is a method of treating a subject with human immunodeficiency virus comprising administering to the subject an effective amount of the composition or agent identified to inhibit immunodeficiency viral transcription. Optionally an effective amount of one or more agents that inhibits a human immunodeficiency viral function can also be administered, for example, those agents described above can be used. Also provided is a method of activating a latent pathogen in a cell, wherein the method comprises contacting the cell with an effective amount of an agent as activating a target cell. Optionally, the latent pathogen is selected from the group consisting of a herpes virus, a hepatitis virus, a mycobacterium, a toxoplasma, and a mycoplasma. Other latent pathogens can also be activated using the disclosed methods, agents and/or compositions. For example, the methods, agents and/or compositions can be used to activate any pathogen that can persist in a subject in a latent state, which refers to the ability of the pathogen to survive intracellularly or extracellularly in a latent state. Such a latent state is typically characterized by the genome of the pathogen persisting, either integrated in the host cell genome or in an episomal form, without producing pathogen proteins at a level that triggers an immune response. Latent pathogens may also persist as inactive pathogens in intracellular or extracellular reservoirs and activation of the intracellular or extracellular environment of the pathogen leads to activation of pathogen gene expression or replication.

The amount of the agent that is administered will vary with the nature of the agent. Any of a variety of methods can be used to determine whether a treatment method is effective. For example, methods of determining whether the methods of the invention are effective in treating an immunodeficiency virus infection are any known test for indicia of immunodeficiency virus infection, including, but not limited to, measuring viral load, e.g., by measuring the amount of immunodeficiency virus in a biological sample, e.g., using a polymerase chain reaction (PCR) with primers specific for an immunodeficiency virus polynucleotide sequence; detecting and/or measuring a polypeptide encoded by an immunodeficiency virus, e.g., p24, gp120, reverse transcriptase, using, e.g., an immunological assay with an antibody specific for the polypeptide; and measuring CD4 cell count in the individual. Methods of assaying an immunodeficiency virus infection (or any indicia associated with an immunodeficiency virus infection) are known in the art, and have been described in numerous publications such as HIV Protocols (Methods in Molecular Medicine, 17) N. L. Michael and J. H. Kim, eds. (1999) Humana Press.

An effective amount of an agent that reactivates latent HIV can be an amount that reactivates latent HIV and reduces the reservoir of latent HIV in a subject during a treatment session by at least about 20%, at least about 30%, at least about 40%, at least about 50%, at least about 60%, at least about 70%, at least about 80%, or at least about 90%. A “treatment session” as used herein can include an administration of a candidate agent to a subject. Such a treatment session can be used in combination with other agents. For example, administration of other chemotherapeutic agents or radiation can be used before, after, or during the session. A “reduction in the reservoir of latent HIV” (also referred to as “reservoir of latently infected cells”) is a reduction in the number of cells in the individual that harbor a latent HIV infection. Whether the reservoir of latently infected cells is reduced can be determined using any known method, including the method described in Blankson et al. (2000) J. Infect. Disease 182(6): 1636-1642. An effective amount of an agent that inhibits HIV-1 transcription can be determined by methods known in the art including detecting a decrease of plasma viral load or an increase in CD4-positive T cells as compared to a control. The control can be the same cells before contact with the agent of can be control cells without contact with the agent.

Thus, an effective amount of a subject agent that reactivates latent HIV can be an amount that activates latent HIV-1 infection in 10², 5×10², 10³, 5×10³, 10⁴, 5×10⁴, 10⁵, or more cells in an individual or a sample, which cells harbor latent HIV.

An effective amount of an agent that inhibits immunodeficiency virus transcription can be an amount that causes a reduction in transcriptional activity in the presence of an immunodeficiency virus activating agent as compared to a control. Methods to monitor transcriptional activity are well known in the art. An effective amount to be used in a subject can also be determined as described above.

An agent or composition can be administered to an individual in combination (e.g., in the same formulation or in separate formulations) with another therapeutic agent (“combination therapy”). The subject agent can be administered in admixture with another therapeutic agent or can be administered in a separate formulation either before, after, or simultaneously with the other therapeutic agent. When administered in separate formulations, a subject agent and another therapeutic agent can be administered substantially simultaneously (e.g., within about 60 minutes, about 50 minutes, about 40 minutes, about 30 minutes, about 20 minutes, about 10 minutes, about 5 minutes, or about 1 minute of each other) or separated in time by about 1 hour, about 2 hours, about 4 hours, about 6 hours, about 10 hours, about 12 hours, about 24 hours, about 36 hours, or about 72 hours, or more.

Other therapeutic agents that can be administered in combination with an effective amount of an agent that inhibits one or more immmunodeficiency virus functions, which functions include, but are not limited to, viral replication; viral protease activity; viral reverse transcriptase activity; viral entry into a cell; viral integrase activity; activity of one or more of Rev, Tat, Nef, Vpr, Vpu, and Vif; and the like.

Therapeutic agents that can be administered in combination therapy, include, but are not limited to, anti-inflammatory, anti-viral, anti-fungal, anti-mycobacterial, antibiotic, amoebicidal, trichomonocidal, analgesic, anti-neoplastic, antihypertensives, anti-microbial and/or steroid drugs. In some embodiments, patients are treated with a subject agent in combination with one or more of the following; beta-lactam antibiotics, tetracyclines, chloramphenicol, neomycin, gramicidin, bacitracin, sulfonamides, nitrofurazone, nalidixic acid, cortisone, hydrocortisone, betamethasone, dexamethasone, fluocortolone, prednisolone, triamcinolone, indomethacin, sulindac, acyclovir, amantadine, rimantadine, recombinant soluble CD4 (rsCD4), anti-receptor antibodies (e.g., for rhinoviruses), nevirapine, cidofovir (Vistide®), trisodium phosphonoformate (Foscarnet®), famcyclovir, pencyclovir, valacyclovir, nucleic acid/replication inhibitors, interferon, zidovudine (AZT, Retrovir®), didanosine (dideoxyinosine, ddI, Videx®), stavudine (d4T, Zerit®), zalcitabine (dideoxycytosine, ddC, Hivid®), nevirapine (Viramune®), lamivudine (Epivir®, 3TC), protease inhibitors, saquinavir (Invirase®, Fortovase®), ritonavir (Norvir®), nelfinavir (Viracept®), efavirenz (Sustiva®), abacavir (Ziagen®), amprenavir (Agenerase®) indinavir (Crixivan®), ganciclovir, AZDU, delavirdine (Rescriptor®), kaletra, trizivir, rifampin, clathiromycin, erythropoietin, colony stimulating factors (G-CSF and GM-CSF), non-nucleoside reverse transcriptase inhibitors, nucleoside inhibitors, adriamycin, fluorouracil, methotrexate, asparaginase and combinations thereof.

The agents or combinations can be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal, intramuscular injection or intravascular injection.

Parenteral administration of the compositions is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

When used in the above or other treatments, a therapeutically effective amount of one of the compounds can be employed in pure form or, where such forms exist, in pharmaceutically acceptable salt form and with or without a pharmaceutically acceptable excipient.

The specific therapeutically effective dose level for any particular subject can depend upon a variety of factors including the disorder being treated and the severity of the disorder; activity of the specific compound employed; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of the compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose.

The dosage can be adjusted by the individual physician in the event of any counterindications. Dosage can vary, and can be administered in one or more dose administrations daily, for one or several days. Guidance can be found in the literature for appropriate dosages for given classes of pharmaceutical products.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter et al., Bioconjugate Chem., 2:447-451, (1991); Bagshawe, K. D., Br. J. Cancer, 60:275-281, (1989); Bagshawe et al., Br. J. Cancer, 58:700-703, (1988); Senter et al., Bioconjugate Chem., 4:3-9, (1993); Battelli et al., Cancer Immunol. Immunother., 35:421-425, (1992); Pietersz and McKenzie, Immunolog. Reviews, 129:57-80, (1992); and Roffler et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Provided herein is a method of making the cell comprising stably transfecting a population of cells with the reporter plasmid infecting said cells with HIV-1 and selecting cells in which the HIV-1 is transcriptionally silent. The reporter plasmid and the replication competent or non-replication competent HIV can be introduced into cells using any known means, including but not limited to, electroporation, calcium phosphate precipitation, infection, and the like. The method of making the cell can further comprise transducing or transfecting the cell with the indicator plasmid. The indicator plasmid can be stably introduced into cells using any known means, including but not limited to, electroporation, calcium phosphate precipitation, infection, and the like. For example, the cell can be retrovirally transduced with the indicator plasmid.

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A. et al., Science, 247, 1465-1468, (1990); and Wolff, J. A. Nature, 352, 815-818, (1991) Such methods are well known in the art and readily adaptable for use with the compositions and methods described herein. The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

The DNA can also be introduced into a cell by electroporation. In this technique, a cell or cells are electroporated in the presence of the desired DNA. Electrical impulses of high field strength reversibly permeabilize biomembranes allowing the introduction of the plasmids. The pores created during electroporation permit the uptake of macromolecules such as DNA. Procedures are described in, e.g., Potter, H. et al., Proc. Nat'l. Acad. Sci. USA 81:7161-7165 (1984); and Sambrook, ch. 16.

The term “lipofection” refers to the introduction of such materials using lipid-based complexes. Methods of incorporating particular nucleic acids into expression vectors are well known to those of skill in the art, but are described in detail in, for example, Sambrook et al., Molecular Cloning. A Laboratory Manual (2nd ed.), Vols. 1-3, Cold Spring Harbor Laboratory, (1989) or Current Protocols in Molecular Biology, F. Ausubel et al., ed. Greene Publishing and Wiley-Interscience, New York (1987), both of which are incorporated herein by reference.

Gene transfer techniques that involve the use of liposomes have been described previously in the art (U.S. Pat. Nos. 5,049,386; 4,946,787; and 4,897,355). General lipofection protocols are also described in the following references: Behr et al. (1989) Proc. Natl. Acad. Sci. (U.S.A.) 86: 6982; Demeneix et al. (1991) Int. J. Dev. Biol. 35: 481; Loeffler et al. (1990) J. Neurochem. 54; 1812; Bennett et al. (1992) Mol. Pharmacol. 41: 1023; Bertling et al. (1991) Biotechnol. Appl. Biochem. 13: 390; Felgner et al. (1987) Proc. Natl. Acad. Sci. (U.S.A.) 84: 7413; Felgner and Ringold (1989) Nature 337: 387; Gareis et al. (1991) Cell. Mol. Biol. 37: 191; Jarnagin et al. (1992) Nucleic Acids Res. 20: 4205; Jiao et al. (1992) Exp. Neurol. 115: 400; Lim et al. (1991) Circulation 83: 2007; Malone et al. (1989) Proc. Natl. Acad. Sci. (U.S.A.) 86: 6077; Powell et al. (1992) Eur. J. Vasc. Surg. 6: 130; Strauss and Jaenisch (1992) EMBO J. 11: 417; and Leventis and Silvius (1990) Biochim. Biophys. Acta 1023: 124. Lipofection reagents are sold commercially (e.g., “Transfectam” and “Lipofectin”). Cationic and neutral lipids that are reportedly suitable for efficient lipofection of nucleic acids include those of Felgner (WO91/17424; WO91/16024). In addition, a combination of neutral and cationic lipid has been shown to be highly efficient at lipofection of animal cells and showed a broad spectrum of effectiveness in a variety of cell lines (Rose et al. (1991) BioTechniques 10: 520. The above lipofection protocols may be adapted for use in the present invention, and the preceding references are therefore incorporated in their entirety.

The plasmid and viral genome can also be transferred into cells by other methods of direct uptake, for example, using calcium phosphate. See, e.g., Graham, F., and A. Van der Eb, Virology 52:456-467 (1973); and Sambrook, ch. 16.

Provided herein is a method of activating a latent immunodeficiency virus in a subject, comprising administering to the subject an effective amount of activating anti-CD28 antibody. “Activating” or “superagonistic” in reference to anti-CD28 antibodies are used interchangeably herein. Activating or superagonistic anti-CD28 antibodies can stimulate T cells causing proliferation of primary resting T cells and IL-2 secretion in the absence of TCR or CD3 stimulation. Moreover, as disclosed herein activating or superagonistic anti-CD28 antibodies can reactivate latent HIV-1 infection in contrast to costimulatory anti-CD28 antibodies.

Antibodies can be administered to a subject in a pharmaceutically acceptable carrier. Suitable carriers and their formulations are described in Remington: The Science and Practice of Pharmacy (19th ed.) ed. A. R. Gennaro, Mack Publishing Company, Easton, Pa. 1995 and are described above. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of antibody being administered.

The antibodies can be administered to the subject, patient, or cell by injection (e.g., intravenous, intraperitoneal, subcutaneous, intramuscular), or by other methods such as infusion that ensure its delivery to the bloodstream in an effective form.

Effective dosages and schedules for administering the antibodies may be determined empirically, and making such determinations is within the skill in the art. Those skilled in the art will understand that the dosage of antibodies administered can vary depending on, for example, the subject receiving the antibody, the route of administration, the particular type of antibody used and other drugs being administered. Guidance in selecting appropriate doses for antibodies is found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

Optionally, the activating anti-CD28 antibody can be administered to the subject with an effective amount of one or more agents that inhibit a human immunodeficiency viral function. The combination therapy can be administered as described above. Optionally, as described above, the agent or agents that inhibit(s)s the human immunodeficiency virus function are selected from the group consisting of a viral replication inhibitor, a viral protease inhibitor, a viral reverse transcriptase inhibitor, a viral entry inhibitor, a viral integrase inhibitor, a viral Rev inhibitor, a viral Tat inhibitor, a viral Nef inhibitor, a viral Vpr inhibitor, a viral Vpu inhibitor, and a viral Vif inhibitor.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1

### High Throughput Screening of Compounds that Reactivate Latent HIV-1 Using EGFP as a Read Out

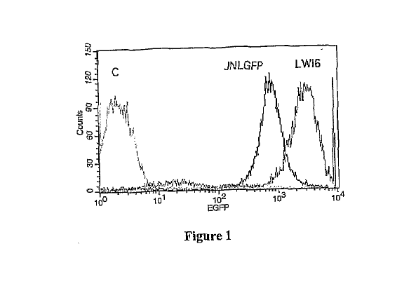

Latently HIV-1 infected reporter cell lines with an increased dynamic range of the EGFP signal. An indicator cell line with HTS feasibility was generated through stable transfection with a reporter plasmid in which the HIV-1 promoter (long terminal repeat; LTR) controls the expression of EGFP. Nucleic acid sequences for HIV-1 LTR (SEQ ID NO:1) can be accessed via GenBank Accession No. X03189. All of the information, including any nucleic acid and amino acid sequences provided for HIV-1 LTR under GenBank Accession X03189 is hereby incorporated in its entirety by this reference. Excision of the HIV-1 C15 LTR from the pU3R-III CAT plasmid (Rosen et al., 1986; Sodroski et al., 1985), using XhoI and HindIII was performed. Using the same restriction enzymes this HIV-1 LTR fragment was inserted into the multiple cloning site of pEGFP1 (Clontech, Palo Alto, Calif.). Jurkat cells, a T cell lymphoma cell line, were then stably transfected with the resulting pLTR-GFP plasmid. Following G418 selection, the cells were cloned based on a low EGFP background signal and then finally selected for their ability to respond to high level of EGFP expression following HIV-1 infection. The final cell line was termed JLTRG cells. In the next step, JLTRG cells were infected with two primary HIV-1 patient isolates (HIV-1 WI70, HIV-1 REJO). The cells that survived the initial infection were cloned by limiting dilution and the resulting clones were screened for the presence of an integrated, but transcriptionally silent copy of the HIV-1 genome that was activated following TNF-α or PMA stimulation. In the resulting latently infected T cell clones (LWI4, LWI6, LWI7, LRJ3, LRJ5, LRJ12), maximal EGFP expression was increased by a factor of 10 in comparison to other reporter cell lines incorporating GFP into the HIV-1 genome when analyzed by flow cytometry (FIG. 1). As a result, these latently infected T cell clones exhibit an up to 10-fold increased dynamic range of EGFP expression when analyzed on a fluorescence plate reader.

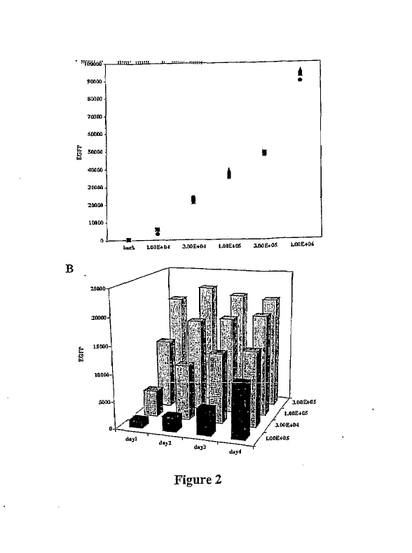

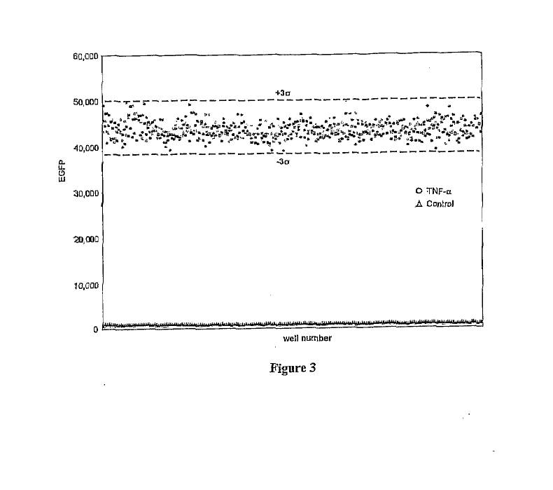

Assay optimization in 334-well format plates. The availability of compounds often can be a limiting factor during the screening of compound libraries. The need to work with low quantities of material and thus low numbers of reporter cells that are treated has to be balanced with the requirements to obtain a sufficiently high signal to background ratio to avoid false negative hits during the screen. To optimize the cell number per well in a 384-well plate format, unstimulated and TNF-α stimulated LWI cells (clone#6) over a wide range of cell concentrations (1×10⁴-1×10⁶ cells/well) were titrated and EGFP fluorescence measured after 24 and 48 h (FIG. 2A). Although EGFP fluorescence continued to increase up to the highest density of cells, wells with 1×10⁵ cells gave a 40-fold increase of EGFP fluorescence intensity over background after 24 h, and an 80-fold increase after 48 h. At this cell density the dynamic range of the assay was sufficiently high to detect small shifts in EGFP expression induced by less potent compounds, thereby minimizing the amount of false negative hits during assay analysis. Using this cell density, a Z′-test was performed using 384-well plates. 1×10⁵ cells in 80 μl of phenol red free RPMI1640 supplemented with 5% FBS were manually seeded into each well of the 384-well plate, and 96-wells in the lower right and the upper left quadrant were treated with TNF-α or PMA. Addition of TNF-α (1 ng/ml) or PMA (5 ng/ml) in a volume of 10 μl was done manually. Automated loading of the plates with cells and compounds in a fully automated screen can further improve results. Twenty-four and 48 h post stimulation, the plates were analyzed for EGFP expression using a BioTek Synergy (Winooski, Vt.). The excitation filter used in the experiments was 465/40 nm, the utilized emission filter was 520/20 nm. The resulting Z′-factors for this cell line when stimulated with TNF-α was determined as Z′=0.85 at the 48 h time point, defining the assay as extremely robust. FIG. 3 depicts a graphic that displays the EGFP fluorescence intensity in arbitrary units as determined from the three different plates that were used to calculated the Z′-factor. The relatively narrow distribution of the data points, which can be significantly increased by automated pipetting on a robotic platform, and the high signal over background demonstrates that the excellent Z′-value of the assay is based on a very high dynamic range and high reliability. The assay thus combines a dynamic range that can be usually only achieved by luciferase assays with the advantages of using EGFP as a read-out: no manipulation during assay preparation or assay analysis.

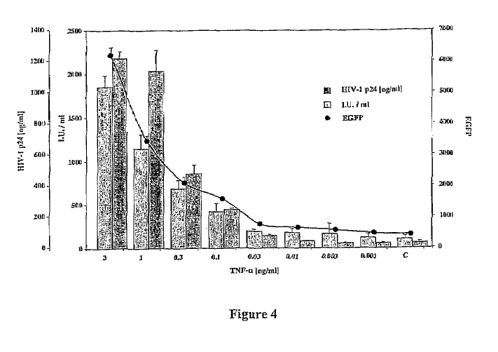

Direct correlation of HIV-1 p24 protein expression, release of infectious viral particles and EGFP fluorescence intensity. Prerequisite for the efficient usage of the cell lines during any HTS effort is the direct correlation of EGFP expression as the surrogate marker of HIV-1 expression, with the production of viral proteins (p24 Gag) and optionally the secretion of infectious viral particles (I.U.). Whether EGFP indeed serves as a direct and quantitative marker of HIV-1 expression by stimulating the cells with various concentrations of TNF-α (0.01-10 ng/ml) was determined. After 48 h, supernatants from each culture were harvested and used to determine the HIV-1 p24 Gag protein content by a commercially available ELISA. Supernatants were further titrated on JC53 cells, an indicator cell line that uses HIV-1 LTR-controlled luciferase production as a read-out, to determine the amount of infectious viral particles released from the LWI cells following reactivation. EGFP fluorescence intensity of the cells was determined by flow cytometry and measured as overall mean channel fluorescence of the culture. The extremely tight correlation between these three markers of HIV-1 expression in LWI cells is depicted in FIG. 4, demonstrating that EGFP indeed serves as an accurate marker of HIV-1 expression over a broad range of HIV-1 expression levels.

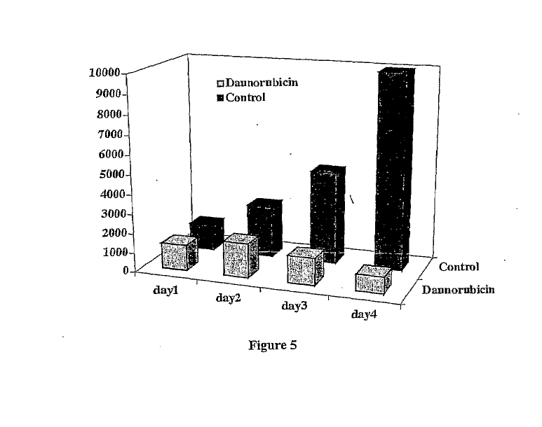

Influence of altered cell proliferation, cell viability and compound auto-fluorescence on EGFP fluorescence intensity. To avoid false positive or false negative hits during the HTS effort, extreme care has to be given to compensate for unspecific changes in the overall fluorescence intensity that are caused by altered levels of cell proliferation, possible changes in cell viability and compound induced changes in the basal fluorescence level. For example, compounds that have fluorochrome characteristics in the same excitation and emission range as EGFP can unspecifically increase the fluorescence signal and can cause false positive hits. On the other hand, too high concentrations of compounds that can reactivate latent HIV-1 infection, but within the chosen concentration range, either inhibit cell proliferation or decrease cell viability can result in false negative hits. To compensate for such effects, an immortalized T cell line was developed that expresses high levels of EGFP under a minimal cytomegalo virus promoter (JEGFP cells) and can be used as a cell line for verification screens. Compounds that unspecifically increase EGFP expression in the latently infected LWI cells due to their fluorochrome characteristics also increase EGFP fluorescence in these cells, and can be detected as false positive hits. Similarly, JEGFP cells can be used to detect changes in the proliferation rate or the viability of the treated cells. As JEGFP cells divide every 24 h and EGFP fluorescence intensity, when measured by plate based fluorometry, directly correlates with the cell density (FIG. 5), any influence of the tested compound on cell proliferation or cell viability can be immediately indicated by a drop in EGFP fluorescence. As an example, 10,000 JEGFP cells were seeded into a well of a 384-well plate and cells were treated with increasing amounts of daunorubicin, an anthracycline antibiotic that is particularly toxic for lymphoma cells, but at low concentrations that are only slightly toxic has some HIV-1 reactivating potential. EGFP fluorescence linearity increased for the first 72 h of the experiments, whereas addition of daunorubicin resulted in a sharp drop in EGFP expression. As the daunorubicin example demonstrates, compounds that cause a drop in EGFP expression in a screen using JEGFP cells can cause inhibition of cell proliferation or cell death and thus have possibly been used at too high concentrations. If indicated, the respective compounds can then be reevaluated on LWI cells for their ability to reactivate latent HIV-1 infection at lower concentrations. By this means, potential false negative hits can be minimized. While reducing the likelihood of false positive or false negative hits during the screening effort by determining potential cytotoxicities of the tested compounds, these assays also provide data for the compounds in vivo use.

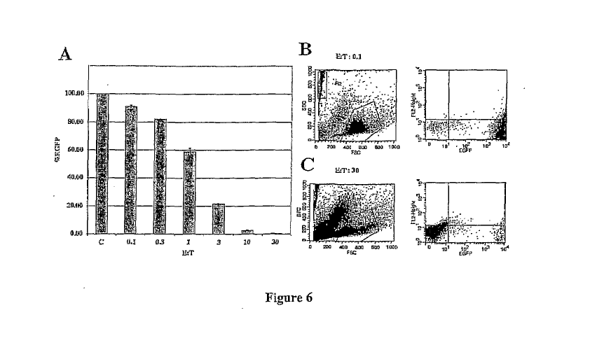

Influence of the tested compounds on the cytotoxic activity of primary CD8-positive T cells. As the immuno-competence of HIV-1 patients can be generally impaired by the infection, treatments that aim at HIV-1 reactivation should avoid further suppression of the immune response, in particular the cytotoxic T cell response. It is thus important to determine potential immunosuppressive characteristics of compounds that reactivate HIV-1 infection at an early stage during drug evaluation. A rapid and simple assay that allows quantification of the capacity of compounds to inhibit the T cell mediated cytotoxic response in the setting of a mixed lymphocyte reaction was developed to account for potential immunosuppressive effects of the tested compounds. For this purpose, stimulated PBMCs from healthy donors, which serve as effector cells, are mixed at different ratios with JEGFP cells, which function as target cells (FIG. 6). Untreated PBMCs efficiently lysed the JEGFP cells, and EGFP expression decreased as a function of the PBMC concentration. Pretreatment of the PBMCs with the compound of interest prior to the mixed lymphocyte reaction then reveals whether the compound influences the cytotoxic capacity of the PBMC population. The assay can be analyzed by plate-based fluorometry or by flow cytometry. Analysis of the assay can be performed after 24 h, which represents the physical half-life of EGFP.

Advanced Confirmation Assays Evaluating the HIV-1 Reactivating Capability.

The final steps of preclinical drug evaluation, quantitative ex vivo testing of the compound's ability to reactivate latent HIV-1 infection in PBMCs derived from HIV-1 infected patients, and comprehensive in vitro and in vivo toxicity profiles are very labor intensive and costly. Before potential drug candidates are moved into this phase of the preclinical evaluation, (a) the quantitative ability of the compound to reactivate HIV-1 in a diverse population of latently infected cells, and (b) the ability of the compound to reactivate latent HIV-1 infection in primary T cells needs to be confirmed.

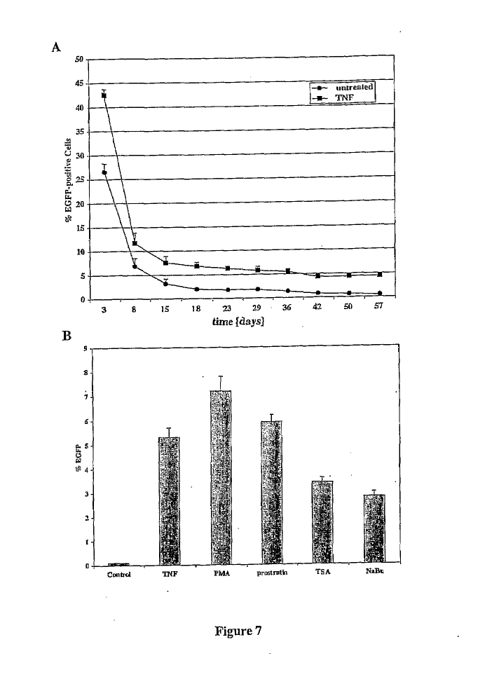

a) Quantification of the HIV-1 reactivating capabilities of test compounds. Following infection, HIV-1 integrates into the cellular genome. Integration is random, although HIV-1 preferentially integrates into euchromatin structures. Latency development can be thus influenced by a multitude of factors that alter gene expression or DNA structure, such as divergent gene expression patterns or the cell cycle stage at the time point of viral integration. Although the reporter cell lines are representative for the majority of latently infected cells and reliably identify most reactivating agents during the screening effort, they can not allow quantification of the capability of the individual compounds to reactivate HIV-1 infection from a diverse population of latently infected cells. To account for the influence that the side of integration of a latent virus can have on a compounds ability to reactivate the virus, a bulk population of latently infected cells (FIG. 7) was established. Following infection of JLTRG cells with HIV-1 NL43, a rapid increase of the level of infection can be observed. On day 3, an average 27% of the cells were productively infected. Interestingly, TNF-α stimulation on day 2 revealed that at this time point, an additional 15% of the cells were non-productively infected. Until day 15, the percentage of productively infected cells rapidly declined to 3% of the overall cell population. At this time point, an additional 5% of the cells were non-productively infected. The rapid decline of the productively infected population was solely caused by the cytotoxic effect of the virus, as no functional inhibition of cell proliferation could be detected by PKH26 staining. Over a total period of 57 days, the size of the productively infected population further declined to 0.2%, but importantly, the size of the non-productively infected cell population remained stable at 5% of the total cell population, indicating that these cells hold a stably integrated transcriptionally silent provirus. Further experiments demonstrated that latent infection develops in 10% of the initial infected cells, independent of the level of initial infection (FIG. 7). Since 1×10⁶ cells were initially infected, and virus mediated inhibition of cell proliferation influences the relative level of infection over time can be excluded, it can be calculated that the latently infected cell population should consist of 5,000 individual latently infected cells. This bulk population of cells allows quantification of the capability of the respective compounds to reactivate latent HIV-1. This was demonstrated by stimulating the cell population with several compounds known to reactivate latent HIV-1 infection. Each compound was titrated over a two-log concentration range to allow for optimal reactivation. The results for the optimal concentration of each compound are depicted in FIG. 7B. Phorbol esters (PMA, prostratin) overall seem to be most potent and reactivated latent HIV-1 infection in 7% or 6% of the cells, respectively. TNF-α (10 ng/ml) reactivated latent HIV-1 infection in 5% of the cells, whereas the DNA histone deacetylase inhibitors trichostatin A (TSA) and sodium butyrate (NaBu) reactivated latent HIV-1 infection in only 3% or 2.5% of the cells, respectively. To achieve an accurate estimate of the differences in the capability of different agents to reactivate HIV-1 infection it is important to run a sufficiently high number of cells. For this flow cytometry based analysis, 100,000 cells viable per sample were counted.

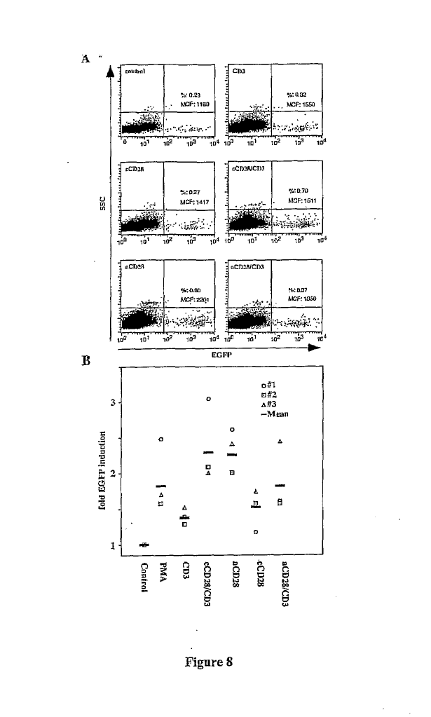

b) Determination of the in vitro HIV-1 reactivating capabilities of compounds in primary T cells. In vitro infection of PBMCs results in the establishment of a substantial population of long-term non-productively or latently infected cells. Following Ficoll-Paque separation of the lymphocyte fraction of blood from healthy donors, the cells were seeded in 6-well plates and stimulated with giant cell medium (IL-4, GM-CSF), as well as with PHA-L and IL-2. Four days following stimulation, the non-adherent cells were removed and infected with an EGFP-expressing indicator virus (Luhder et al., J. Exp. Med. 197:955-966 (2003)) for two hours and then added back to the original wells. The initial infection level on day 4 post infection ranged from 5 to 10% of the total cell population. The cell were then cultured for 10 to 14 days in RPMI supplemented with 10% FBS, in the absence of IL-2. At this time point, the level of productively infected cells had dropped to 0.1 to 0.3%. Stimulation of the non-adherent cells with IL-2, anti-CD3, co-stimulatory anti-CD28 antibody or combinations thereof or superagonistic anti-CD28 antibody revealed that a substantial population (up to 1%) of cells harbored transcriptionally silent viruses, which probably reflect latent HIV-1 infection, that was reactivated by the treatment (FIG. 8). The level of latent infection was dependent on the initial infection level. Usually latency developed in 5% of the initially infected cells. This relatively inexpensive experimental setup allowed accurate determination of the capability of individual compounds to reactivate latent HIV-1 infection in comparison to established reactivating agents by determining the percentage of EGFP expressing cells prior and post stimulation using flow cytometry. At the same time, the experiments provided first information how the tested compounds influence viability of the primary lymphocytes and can be used to determine the resulting cytokine profile, an indicator of potential detrimental effects in the in vivo situation (cytokine release syndrome).

### Example 2

### Efficient Reactivation of Latent HIV-1 Infection by Superagonistic Anti-CD28 Antibodies

Cell culture and reagents. The latently HIV-1 infected T cell line J89GFP was maintained at a density of 0.5×10⁶ cells/ml in RPMI 1640 supplemented with 2 mM L-glutamine, 100 U/ml penicillin, 100 μg/ml streptomycin and 10% heat inactivated fetal bovine serum. Kutsch et al., J. Virol. 76:8776-8786 (2002). The cells were grown up to a density of 1×10⁶/ml and then seeded at 2×10⁵ cells per 96 well in a final volume of 200 μl Peripheral blood mononuclear cells (PBMCs) were isolated from the blood of healthy donors by Ficoll-Paque density gradient centrifugation and were cultured in RPMI 1640 supplemented with 10% heat-inactivated FBS, 2 mM L-glutamine, 100 U/ml of penicillin, and 100 μg/ml of streptomycin. Thymocytes from HIV-1 infected SCID-hu (Thy/liv) mice were isolated as described elsewhere (Brooks et al., Nat. Med. 7:459-464 (2001)), but not CD8 depleted prior to stimulation and analysis.

The following antibodies were used: Anti-CD2 (clone RPA-2.10; Pharmingen, San Diego, Calif.), anti-CD3 (clones UCHT1 and HIT3a; Pharmingen, San Diego, Calif.) and anti-CD28 (clone CD28.2, Pharmingen, San Diego, Calif.; clone L293, Becton&Dickinson, Franklin Lakes, N.J.; clone 5D10, Ancell). The phorbol ester 13-phorbol-12-myristate acetate (PMA), the histone deacetylase inhibitor sodium butyrate (NaBu), and proteinG coated 96 well plates were obtained from Sigma, (St. Louis, Mo.). Recombinant human IL-2 and TNF-α were obtained from R&D Systems (Minneapolis, Minn.). HIV-1 NLENG1-IRES and NLENY1-IRES were derived from HIV-1 NLENG1. Kutsch et al., J. Virol. 76:8776-8786 (2002). An internal ribosome entry site (IRES) was inserted upstream of the nef gene, conferring wildtype Nef-protein expression levels in the infected cells. Levy et al., Proc. Nat. Acad. Sci. 101:4204-4209 (2004). PBMCs were stimulated with the various antibodies at a concentration of 1.0 μg/ml per 1×10⁶ cells for 48 h and then infected with NLENG1-IRES at an MOI of 0.01.

Quantification of cell proliferation and determination of HIV-1 infection. To determine the level of cell proliferation in PBMCs following stimulation with the various T cell specific antibodies used in the experiments, a defined number of SPHERO blank calibration particles (Pharmingen, San Diego, Calif.) were added to each individual culture prior to antibody stimulation. Due to differences in size and granularity, SPHERO blank calibration particles (diameter 6-6.4 μm) can be easily distinguished from PBMCs in a FSC/SSC dot plot analysis using flow cytometry. At the time point of analysis, the flow cytometer was set up to acquire a defined amount of beads, while acquiring all cells in the live gate. The ratio of beads to cells in each culture then allowed for the determination of relative cell proliferation as a result of stimulation by the respective antibodies, in comparison to unstimulated cells. By quantifying the percentage of EGFP-positive cells, this method allowed simultaneous determination of cell proliferation and the level of HIV-1 infection in each culture.

Isolation of thymocytes from SCID-hu (Thy/Liv) mice. Thy/Liv implants from the infected mice were harvested 2-4 weeks post infection and single cell suspensions generated by dissociation of the tissue. Thymocytes were isolated by Ficoll-Paque density gradient centrifugation and were cultured for two days in RPMI 1640 supplemented with 10% heat-inactivated FBS, 2 mM L-glutamine, 100 U/ml of penicillin, and 100 μg/ml of streptomycin, in the presence of indinavir and AZT, to inhibit de novo infection. Productively infected thymocytes, which expressed EYFP, were then removed from the cultures by FACS sorting. Purity of the resulting EYFP-negative cell population was determined by flow cytometric analysis for EYFP and was usually greater than 99.9%. Immediately following the sort, cells were stimulated. HIV-1 reactivation, as indicated by the de novo expression of EYFP was quantified by flow cytometry, acquiring at least 1×10⁶ cells per sample in the live gate.

In vitro generation and analysis of HIV-1 latency in PBMCs. PBMCs were isolated by Ficoll Paque centrifugation, plated into 6-well plates and cultured in RPMI 1640 supplemented with PHA-L (2 μg/ml) and 10% GCT conditioned media (Fisher, Hampton, N.H.). Five days following stimulation the cells were infected with HIV-1 NLENG1-IRES. Levy et al., Proc. Nat. Acad. Sci. 101:4204-4209 (2004). Three days post infection indinavir was added to the culture to inhibit de novo infection. In the absence of IL-2, the cells were cultured for seven days following infection and then subjected to fluorescence activated cell sorting (FACS) to remove EGFP positive, HIV-1 expressing cells. The EGFP negative cell fraction was then activated with the indicated stimuli and reactivation of latent HIV-1 expression was measured 24 h post stimulation by determining EGFP fluorescence using FACS analysis.

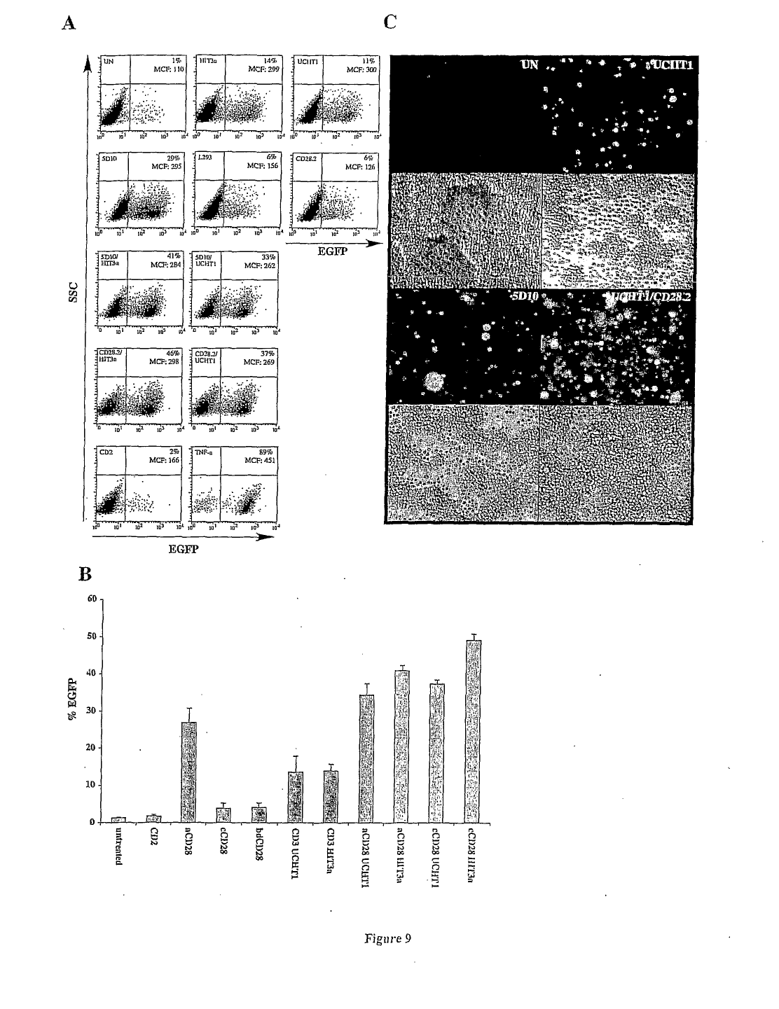

Reactivation of latent HIV-1 infection by anti-CD28 antibodies. When the T cell reporter line (J89GFP) was used to screen the ability of various T cell specific antibodies to reactivate latent HIV-1 infection following a single antibody treatment (FIG. 9). An activating anti-CD28 antibody (clone 5D10) was found to be most potent.

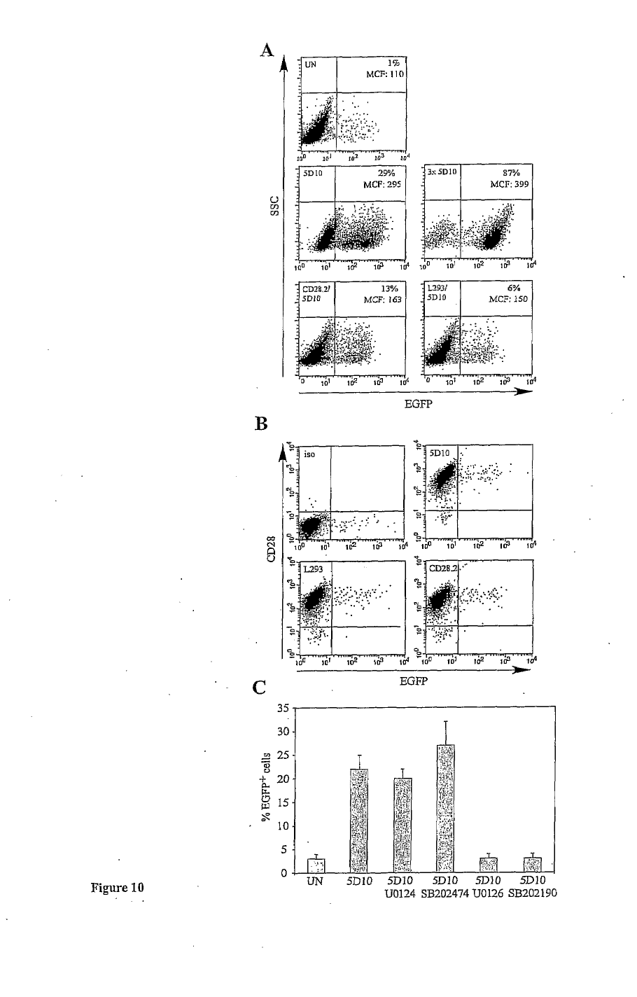

On average, 1% of the untreated J89GFP cells exhibited spontaneous reactivation, as indicated by the expression of EGFP. Anti-CD3 antibodies (UCHT1, HIT3a) reactivated latent HIV-1 infection in J89GFP cells, but despite uniform levels of CD3 expression on all cells, reactivation of latent HIV-1 infection was only observed in 10-15% of the population. Costimulatory anti-CD28 antibodies (clone CD28.2 and L293) reactivated HIV-1 infection in up to 5% of the cells. In contrast, a single treatment with an activating anti-CD28 antibody (clone 5D10) efficiently reactivated latent HIV-1 infection in 30% of the cells in the absence of TCR/CD3 stimulation (FIGS. 9A and 9B). Due to syncytia formation in the culture, which cannot be accounted for using flow cytometry, the determined level of reactivation actually underestimates the actual level of reactivation by an estimated 10-15% (FIG. 9C). Costimulation of CD3 and CD28 resulted in reactivation of latent HIV-1 in 30-50% of the J89GFP cells. Combinations of anti-CD3 antibodies and activating anti-CD28 antibodies were found to be less efficient (30-40% reactivation) than combinations of anti-CD3 antibodies and costimulatory anti-CD28 antibodies (40-50% reactivation) (FIGS. 9A and 9B). Antibody stimulation of the cells under cross-linking conditions in protein-G-coated plates did not result in increased levels of HIV-1 reactivation, but repeated application of anti-CD28 antibodies, in the presence AZT and indinavir to avoid reactivation by de novo infection, resulted in full reactivation of latent HIV-1 on a population basis (FIG. 10A). Pretreatment of J89GFP cells with the costimulatory anti-CD28 antibody L293 abrogated the effect of activating anti-CD28 antibody, proving that indeed CD28 binding and no thus far undetected cross-reactivity of the activating anti-CD28 antibody was responsible for the observed effect (FIG. 10A). An isotype matched control antibody had no effect on the ability of activating anti-CD28 antibody to reactivate latent HIV-1 infection. The functional difference between the anti-CD28 antibodies with respect to reactivation of latent HIV-1 infection cannot be explained by a greatly decreased ability of clones CD28.2 or L293 to bind to the cells, as FACS staining reveals that all three anti-CD28 antibodies efficiently stained the cells (FIG. 10B). As T cell activation with activating anti-CD28 antibodies is controlled by the MAPK pathway (Bischof et al., Eur. J. Immunol. 30:876-882 (2000); Rodriguez-Palmero et al., Eur. J. Immunol. 29:3914-3924 (1999); Tacke et al., Eur. J. Immunol. 27:239-247 (1997)), it was tested whether this pathway was also involved in reactivation of latent HIV-1 in J89GFP cells by activating anti-CD28 antibodies. J89GFP cells were pretreated for one hour with either the p38 MAPK inhibitor SB202190 (1 μM) or the ERK MAPK inhibitor U0126 (1 μM), or with similar concentrations of the inactive control compounds U0124 and SB202474, respectively, and then the cells were stimulated with activating anti-CD28 antibody. Both inhibitors efficiently abrogated the activating effect of the antibody and blocked HIV-1 reactivation (FIG. 10C), demonstrating the importance of the MAP kinase pathway for CD28 mediated HIV-1 reactivation.

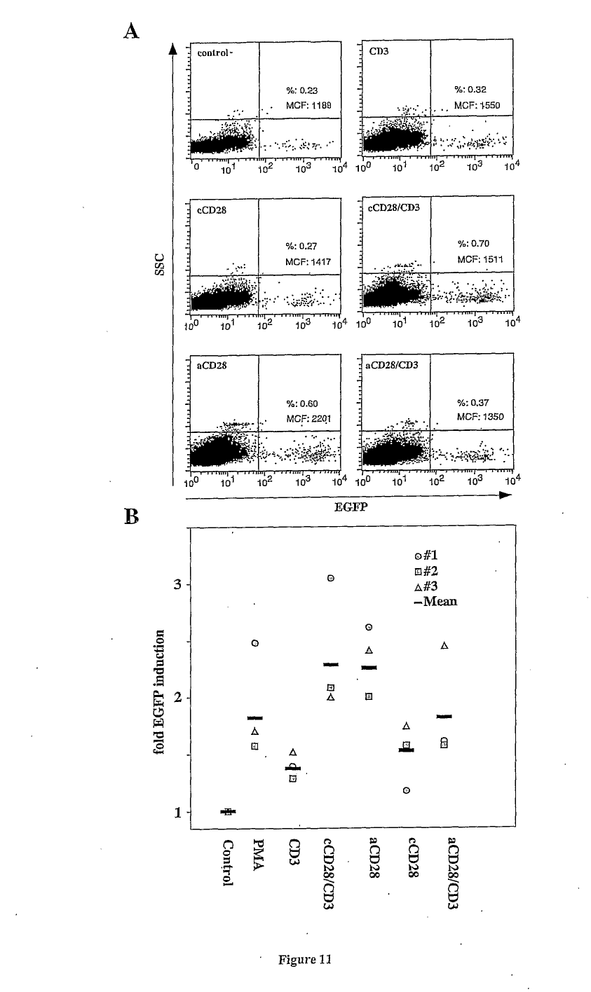

Reactivation of latent HIV-1 infection by anti-CD28 antibodies in human PBMCS. PBMCs from three different donors were infected with replication competent HIV-1 NLENG1-IRES. Three days post infection HIV-1 inhibitors (indinavir/AZT) were added to the cultures to stop de novo infection. At this time point, the infection level varied between 5 and 7%. Seven days following infection, EGFP⁺ cells, were removed by fluorescence activated cell sorting. The purity of the EGFP⁻ fraction was assessed immediately after the sort and was generally >99.9%. The EGFP⁻ cells were then stimulated with the phorbol ester 13-phorbol-12-myristate acetate (PMA), anti-CD3 antibody (UCHT1) or anti-CD3/CD28 antibody (UCHT1/CD28.2) as positive controls, as well as with activating anti-CD28 antibody (5D10) and co-stimulatory anti-CD28 antibody (CD28.2) (FIG. 11). Twenty-four hours post stimulation levels of HIV-1 reactivation were determined as the percentage of EGFP⁺ cells. Representative data are depicted in FIG. 11A. In this culture the initial infection level was 7%. Twenty-four hours following the cell sorting procedure, a low level of spontaneous HIV-1 reactivation was observed in the untreated control cultures. With a background of 0.23% of the cells being EGFP⁺ in the untreated control culture, anti-CD3/CD28 costimulation was most potent to reactivate latent HIV-1 infection (0.70% EGFP⁺). Under the assumption that this antibody combination reactivates HIV-1 infection in all latently infected cells, latency would have developed in 10% of the initially infected cells. Activating anti-CD28 antibody (5D10) treatment and PMA stimulation induced HIV-1 reactivation in 0.60% of the cells. Anti-CD3 antibody, surprisingly, had only a minor effect on the pool of latently infected cells (0.27% EGFP), a result that was consistent in all three donors. Costimulation with an activating anti-CD28/anti-CD3 antibody combination surprisingly was found less efficient to promote HIV-1 reactivation (0.37% EGFP⁺), again a finding that was consistent in all three donors (FIG. 11B).

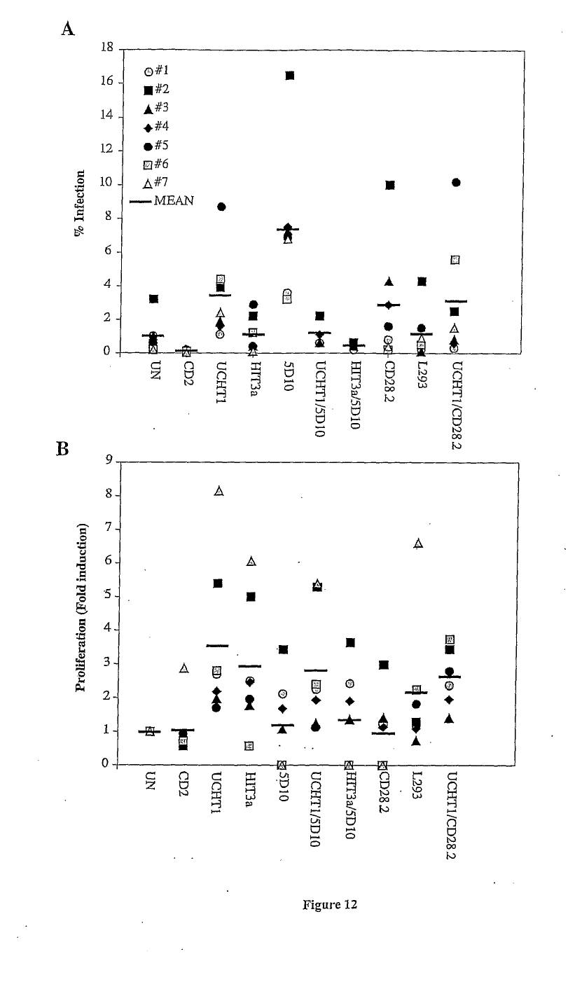

Susceptibility of PBMCs to HIV-1 infection following stimulation with various T cell specific antibodies. Although HAART efficiently suppresses HIV-1 replication, a general concern during all attempts to reactivate latent HIV-1 from its reservoirs has to be whether and to what extent the chosen strategy promotes HIV-1 replication and increases the likelihood of de novo infection. To study the ability of the studied T cell-specific antibodies to promote HIV-1 replication, PBMCs were stimulated with the antibodies/antibody combinations indicated in FIG. 11 and infected the cells with a reporter virus. After 5 days, levels of cell proliferation and infection efficiency in PBMCs from seven donors were determined by flow cytometry. Similar to its ability to reactivate latent HIV-1 infection, we found that the activating anti-CD28 antibody 5D10 by itself promoted HIV-1 replication more efficiently than anti-CD3 antibodies. Whereas simultaneous stimulation with anti-CD3 and costimulatory anti-CD28 antibodies promoted HIV-1 replication, combinations of anti-CD3 antibodies and activating anti-CD28 antibodies abrogated HIV-1 replication. (FIG. 12).

The baseline infection level in unstimulated PBMCs was on average 1% (0.2-3.2%). The results following stimulation of CD3 were antibody-dependent: Following stimulation with UCHT1 an average of 3.4% (1.1-8.7%) of the cells were infected, whereas HIT3a stimulation did not render the cells susceptible to HIV-1 infection. Both anti-CD3 antibodies had a comparable effect on cell proliferation (3-fold induction). Stimulation of the PBMCs with activating anti-CD28 antibody (5D10) allowed for the infection of 7.4% (3.6-16.5%) of the cells, but did not result in a significant increase in cell proliferation. If the sample with the highest rate of infection is excluded from the calculation, mean infection level would still be 5.9%. Stimulation with the costimulatory anti-CD28 antibody CD28.2 also raised the levels of infectivity (2.9%; 0.2-10%), but had no effect on cell proliferation. The second costimulatory CD28 antibody L293 neither increased the susceptibility of the cells to HIV-1 infection, nor promoted proliferation. Costimulation of the cells with CD3-UCHT1 and costimulatory CD28.2 or CD3-HIT3a and costimulatory CD28.2 combinations, with respect to infection and proliferation, reproduced the data obtained for stimulation with CD3-UCHT1 or CD3-HIT3a alone. Costimulation of PBMCs using activating anti-CD28 antibody 5D10 and CD3-UCHT1 or 5D10 and CD3-HIT3a, respectively, abrogated the promoting effect of the activating anti-CD28 antibody with respect to infection. At least in the case of the 5D10/UCHT1 antibody combination, stimulation still resulted in cell proliferation.

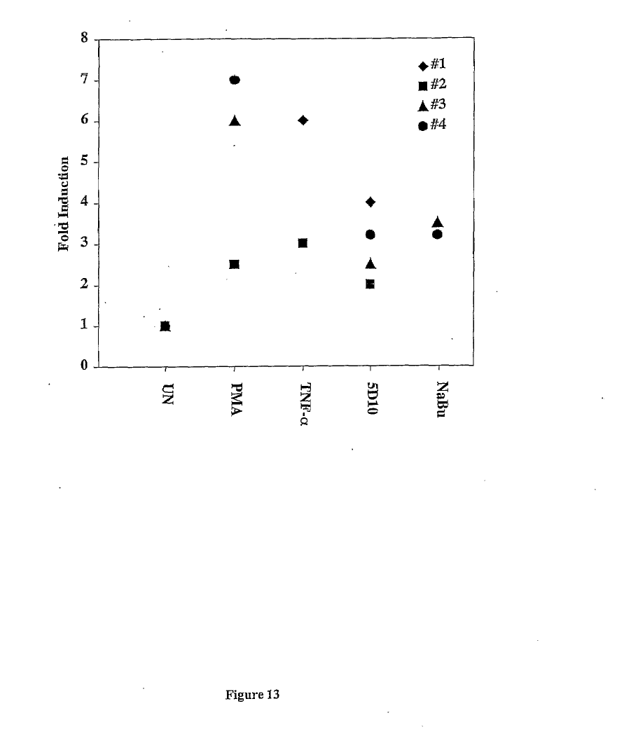

Reactivation of latent HIV-1 infection by anti-CD28 antibodies in human thymocytes derived from SCID-hu (Thy/liv) mice. Whether activating anti-CD28 antibody would also reactivate latent HIV-1 in a system where latency in primary cells was achieved under in vivo conditions was tested. For this purpose, a modification of the HIV-1 established SCID-hu (Thy/Liv) mouse system was used that has been previously used for the study of HIV-1 latency. Brooks et al., Nat. Med. 7:459-464 (2001). SCID-hu (Thy/Liv) mice were infected with HIV-1 NLENY1-IRES to allow for the easy and direct detection of infected cells using flow cytometry. Levy et al., Proc. Nat. Acad. Sci. 101:4204-4209 (2004). 2-4 weeks following the initial infection, the mice were sacrificed and thymocytes isolated. Levels of infection were determined by flow cytometry for EYFP, and varied in the different animals between 0.5 and 6% of the total thymocyte population. Cells were cultured for two days in the presence of a reverse transcriptase (AZT) and a protease inhibitor (indinavir) to inhibit de novo infection, but allow for recently integrated virus to express EYFP. The cultures were then subjected to fluorescence activated cell sorting to remove all EYFP positive cells. Immediately following the cell sorting procedure, the cells were stimulated with activating anti-CD28 antibody (clone 5D10), an isotype control antibody, and as positive controls with TNF-α (Butera et al., J. Virol. 65:4645-4653 (1991)) or the phorbol ester PMA. Folks et al., J. Immunol. 140:1117-1122 (1988). Thymocytes from two animals were also treated with the histone deacetylase inhibitor sodium butyrate. Golub et al., AIDS 5:663-668 (1991); Laughlin et al., Virology 196:496-505 (1993); Quivy et al., J. Virol. 76:11091-11103 (2002). Unstimulated cells were cultured to determine the level of latently infected cells that would spontaneously exhibit HIV-1 reactivation. Accuracy of the results was assured by running at least 1×10⁶ cells per treatment condition. Prior to cell sorting 1.71% of the thymocyte population expressed EYFP as a marker for HIV-1 infection. Twenty-four hours after the depletion of the EYFP⁺ cell population, 50 of 1×10⁶ cells in the untreated culture and the culture treated with an isotype matched antibody exhibited an EYFP⁺-phenotype, indicating spontaneous reactivation. Cultures treated with TNF-α or PMA produced 250, respectively 260 EYFP⁺ cells. These numbers indicate that latency had developed in about 1% of all infected cells. Stimulation of the cultures with activating anti-CD28 antibody resulted in HIV-1 reactivation in 150 cells, or 60% of the level of PMA or TNF-α-mediated reactivation. The four sampled animals all received implants from different fetal donors to account for potential genetic differences in the donor cells that would influence susceptibility to the treatment. Overall, PMA induction of latent HIV-1 infection in thymocytes varied between 2.5-7-fold and TNF-α induction between 3-6-fold among the tissue samples, while activating anti-CD28 antibodies induced a 2.5-4-fold increase in the level of HIV-1 expression from latency. A similar level of reactivation was also seen following stimulation with the histone deacetylase inhibitor sodium butyrate (FIG. 13).

### Example 3

### High Throughput Screening of Compounds that Inhibit HIV-1 Transcription Using EGFP as a Read Out

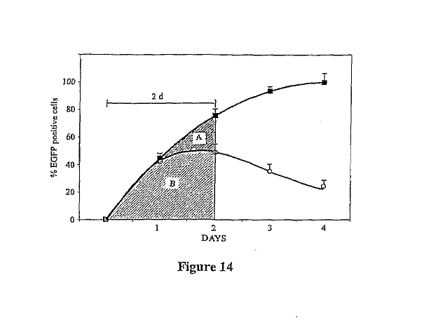

The latently HIV-1 infected reporter T cell line J89GFP (Kutsch et al., 2002) was pretreated with the respective compound and then stimulated with various concentrations of TNF-α. As reactivation of latent HIV-1 is dependent on Tat activity, a sufficiently potent Tat inhibitor would suppress HIV-1 reactivation as indicated by an increase in EGFP fluorescence. FIG. 14 demonstrates that the established Tat inhibitor Ro24-7429 is not capable of suppressing reactivation of latent HIV-1 infection in the presence of 1 ng/ml TNF-α. During the first 24 h, the Tat inhibitor had no influence on the level of HIV-1 expression. Levels of reactivation with respect to the percentage of HIV-1 positive cells, as well as the EGFP MCF intensity were identical. Whereas EGFP expression in TNF-α treated J89GFP in the absence of Ro24-7429 continued to increase until 96 h post stimulation, EGFP MCF peaked after 48 h, reaching 50% of the maximal EGFP MCF seen in the absence of Ro24-7429, and the slowly declined again (FIG. 14). The observed increase of EGFP fluorescence in TNF-α treated J89GFP cells can be described as an invasion kinetics of the first order, whereas the kinetics of EGFP expression following TNF-α mediated reactivation of HIV-1 in the presence of Ro24-7429 can be best described by a Bateman function, that describes onset and elimination of HIV-1 expression. Again, integration of the equation describing each curve allows for the determination of the relative amount of virus being produced in the critical 2 days time frame. Area (A+B) represents the amount of virus produced following reactivation, and Area B represents the amount of virus produced in the presence of Ro24-7429. Assuming that HIV-1 reactivation and HIV-1 expression following integration during an acute infection follow a similar kinetic pattern, the results confirm the conclusion from the chronically active infected cells and demonstrates that during the important initial 2 days period, Ro24-7429 only inhibits 20% of the virus production.

To transfer this assay on a plate based format amenable to High Throughput Screening, several novel latently infected reporter cell lines were developed, which, in comparison to the J89GFP cell lines, exhibit greatly increased levels of EGFP expression upon reactivation and are fully amenable for HTS. One such cell line is the LWI6 cell line, which was generated as described in example 1 above.

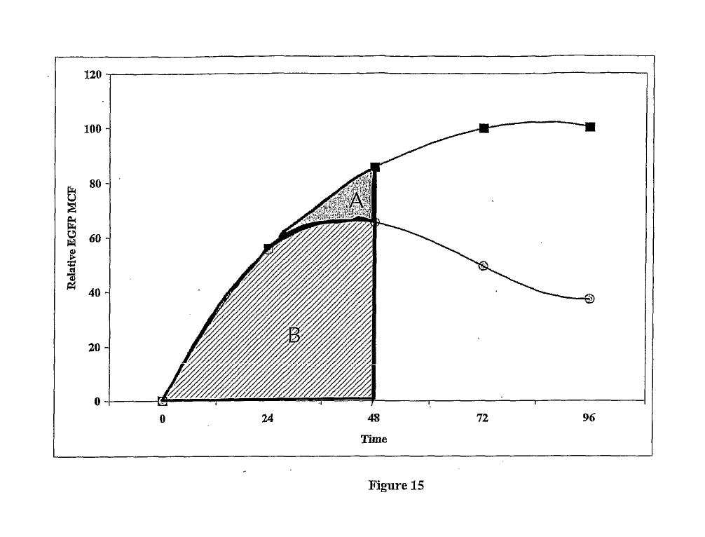

For experiments using flow cytometric analysis to determine the level of EGFP expression at the various time points of the kinetic experiment, LWI6 cells were maintained in RPMI 1640 supplemented with 5-10% FBS. LWI6 cells were seeded at a density of 1×10⁶ cells/ml in a 24 well plate at a total cell number of 1×10⁶ cells per well. For experiments in a plate based format using a fluorescent plate reader the cells were cultured in RPMI 1640 without phenol red and supplemented with 2% FBS. The cells were seeded into the wells of a 384 well plate at a cell density of 1×10⁶ cells/ml in a total volume of 100 μl. Where indicated, the cells were the pretreated for 6 h with the HIV-1 transcription inhibitor Ro24-7429 and then stimulated with TNF-α (1 ng/ml). EGFP expression following TNF-α (1 ng/ml) mediated reactivation of latent HIV-1 infection in LWI6 cells in the presence or absence of Ro24-7429 (10 μM) was then measured at the indicated time points by either determining the mean channel fluorescence and the percentage of EGFP expressing cells using flow cytometric analysis or by determining total fluorescence in the individual wells using plate based fluorometry. As shown in FIG. 15, following stimulation, area (A+B) represents the total amount of virus produced over a two day period of time, whereas area B represents the amount of virus produced in the presence of Ro24-7429. Results represent the mean ±S.D. of three independent experiments.

### Example 4

### High Throughput Screening of Compounds that Reactivate Latent HIV-1 Using EGFP as a Read Out and Using a Cytotoxicity Marker

A latently HIV-1 infected reporter cell line was developed in which HIV-1 expression is indicated by EGFP fluorescence and in which an alteration in the fluorescence level of a spectrally separate second fluorescent protein that is constitutively expressed at high levels (DsRedExpress) is used to determine potential compound cytotoxicities. This cell line decreased the overall drug screening effort, as no additional cytotoxicity screen needed to be performed. These reporter cells were termed LWI6-R.

To obtain LWI6-R cells (R for red fluorescence), LWI6 cells were retrovirally transduced with pMSCV-DsRedExpress. One non-limiting example of a vector that can be used to create pMSCV-DsRedExpress is pMSCVpuro, which is available from Clontech Laboratories, Inc., Mountain View, Calif. pMSCV-DsRedExpress was co-transfected with pHIT60 and pVSVG, two vectors coding for the structural proteins of murine leukemia virus and the vesicular stomatitis virus G-protein into 293T cells. The supernatants from these cultures were harvested after 48 h and used to transduce LWI6 cells. The cell culture was then sorted for high expression of DsRedExpress and the resulting population was termed LWI6-R cells.

When titrated into a 384 well plate as few as 1×10⁴ cells per well were detected using a plate-based fluorescent reader (BIOTEK Synergy HT; Filters: excitation: 530/35 nm; emission: 590/20 nm). At 2.5×10⁵ LWI6-R cells per well, the target cell number commonly used in the reporter assays, a 10-fold signal induction over non-transduced LWI6 control cells was measured. The signal improved if an available filter set was optimized to measure DsRedExpress (excitation: 557 nm; emission: 579 nm).

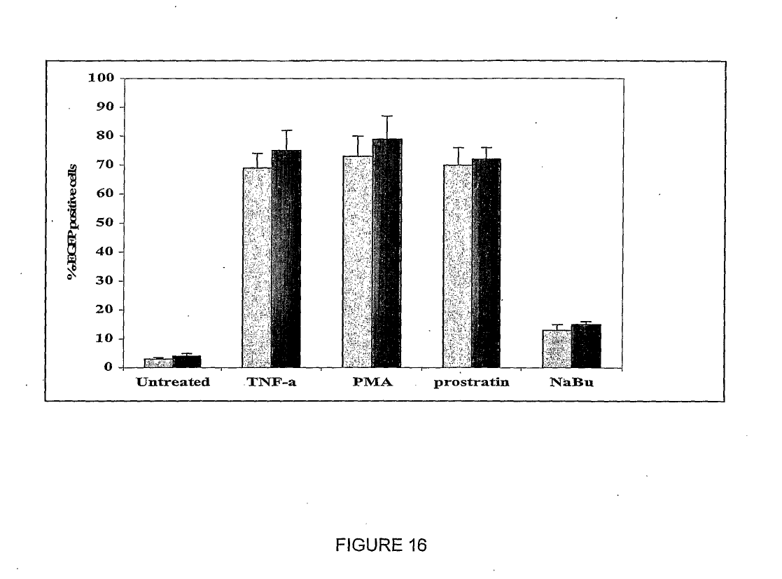

That the LWI6-R system reproduces data obtained with the LWI6 cells was demonstrated by testing several activators of latent HIV-1 (NaBu, prostratin, PMA, TNF-a) and the response of the two cell types was compared with respect to the level of HIV-1 reactivation as indicated by the level of EGFP expression at the population level (FIG. 16).

The use of DsRedExpress as a quantitiative viability marker was further demonstrated in a manually performed drug screen of a 2,000 compound library. After obtaining the fluorescence intensities for EGFP and DsRedExpress at 48 h the data were analyzed, and autofluorescent, toxic and activating compound groups were identified.

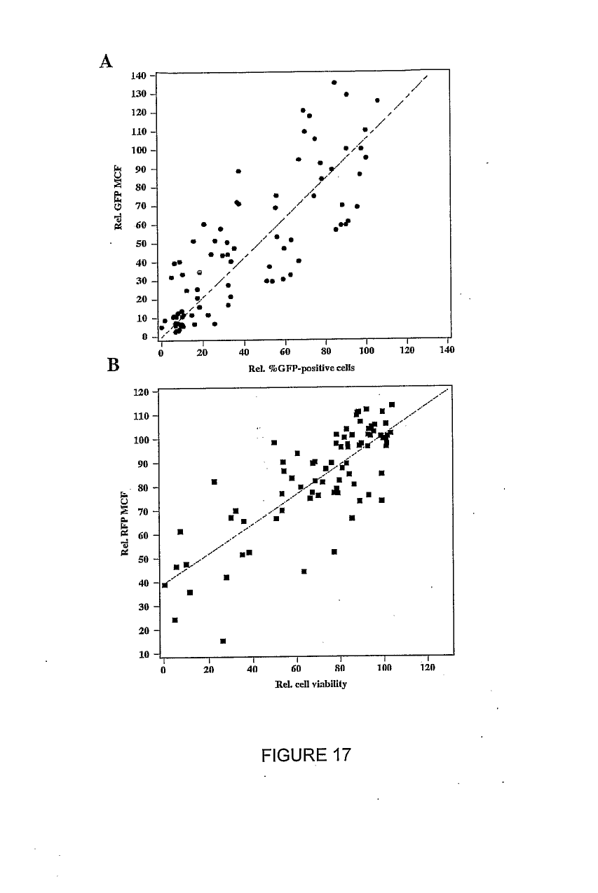

A selection of 86 compounds that was biased for the above three compound groups was selected and subjected to flow cytometric analysis to determine the percentage of cells exhibiting EGFP expression as a measure for reactivation activity. The relative percentage of EGFP-positive cells (normalized to the maximum percentage of EGFP-positive cells obtained in PMA treated cultures) was plotted against the relative GFP expression as determined by plate-based flow cytometry (normalized against maximum fluorescence in untreated co-cultures) (FIG. 17A). The linear curve fit is slightly biased towards EGFP as measured by plate-based fluorometry, as flow cytometry fails to acquire syncytia, which are disrupted, but are accounted for in a plate-based set-up.

Relative DsRedExpression as a marker for cell viability was measured by plate-based fluorometry (normalized against maximum fluorescence in untreated co-cultures) and plotted against relative cell viability as determined by flow cytometry (FIG. 17B). Cell viability was calculated using FSC/SSC (Forward scatter/Side scatter) dotplot data. For each of the samples the number of cells in the life gate was divided by the total number of cells acquired and normalized against samples obtained from untreated cultures. The resulting linear curve fit indicates that cell death in the plate-based format is underestimated at the 48 h time-point by 40%, because of the long half-life of DsRedExpress (>24 h). However, although the dynamic range of the DsRedExpress marker is reduced compared to flow cytometric analysis, it is a reliable and sufficiently sensitive marker of cell death in a HTS set-up, and improved results are obtained by extending the drug screen to 72 h.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### REFERENCES

Abramowicz, D., A. Crusiaux, and M. Goldman. 1992. Anaphylactic shock after retreatment with OKT3 monoclonal antibody. N. Engl. J. Med. 327:736.
Abramowicz, D., and M. Goldman. 1996. Efficacy of rejection prophylaxis with OKT3. Transplantation 62:700-701.
Abramowicz, D., L. Schandene, M. Goldman, A. Crusiaux, P. Vereerstraeten, L. De Pauw, J. Wybran, P. Kinnaert, E. Dupont, and C. Toussaint. 1989. Release of tumor necrosis factor, interleukin-2, and gamma-interferon in serum after injection of OKT3 monoclonal antibody in kidney transplant recipients. Transplantation 47:606-608.
Alegre, M. L., K. Gastaldello, D. Abramowicz, P. Kinnaert, P. Vereerstraeten, L. De Pauw, P. Vandenabeele, M. Moser, O. Leo, and M. Goldman. 1991. Evidence that pentoxifylline reduces anti-CD3 monoclonal antibody-induced cytokine release syndrome. Transplantation 52:674-679.
Bednarik, D. P., Cook, J. A., and Pitha, P. M. a., 1990. Inactivation of the HIV LTR by DNA CpG methylation: evidence for a role in latency. Embo J 9(4), 1157-1164.
Bischof, A., T. Hara, C. H. Lin, A. D. Beyers, and T. Hunig. 2000. Autonomous induction of proliferation, JNK and NF-kB activation in primary resting T cells by mobilized CD28. Eur. J. Immunol. 30:876-882.
Bounou, S., N. Dumais, and M. J. Tremblay. 2001. Attachment of human immunodeficiency virus-1 (HIV-1) particles bearing host-encoded B7-2 proteins leads to nuclear factor-kappa B- and nuclear factor of activated T cells-dependent activation of HIV-1 long terminal repeat transcription. J. Biol. Chem. 276:6359-6369.
Brodie, S. J. 2000. Nonlymphoid reservoirs of HIV replication in children with chronic-progressive disease. J. Leukoc. Biol. 68:351-359.
Brooks, D. G., P. A. Arlen, L. Gao, C. M. Kitchen, and J. A. Zack. 2003. Identification of T cell-signaling pathways that stimulate latent HIV in primary cells. Proc. Natl. Acad. Sci. USA 100:12955-12960.
Brooks, D. G., D. H. Hamer, P. A. Arlen, L. Gao, G. Bristol, C. M. Kitchen, E. A. Berger, and J. A. Zack. 2003. Molecular characterization, reactivation, and depletion of latent HIV. Immunity 19:413-423.
Brooks, D. G., S. G. Kitchen, C. M. Kitchen, D. D. Scripture-Adams, and J. A. Zack. 2001. Generation of HIV latency during thymopoiesis. Nat. Med. 7:459-464.
Brooks, D. G., and J. A. Zack. 2002. Effect of latent human immunodeficiency virus infection on cell surface phenotype. J. Virol. 76:1673-1681.
Bukczynski, J., T. Wen, and T. H. Watts. 2003. Costimulation of human CD28− T cells by 4-1BB ligand. Eur. J. Immunol. 33:446-454.
Bushman, F. D., and Craigie, R., 1992. Integration of human immunodeficiency virus DNA: adduct interference analysis of required DNA sites. Proc Natl Acad Sci USA 89(8), 3458-3462.
Butera, S. T., V. L. Perez, B. Y. Wu, G. J. Nabel, and T. M. Folks. 1991. Oscillation of the human immunodeficiency virus surface receptor is regulated by the state of viral activation in a CD4+ cell model of chronic infection. J. Virol. 65:4645-4653.
Butera, S. T., 2000. Therapeutic targeting of human immunodeficiency virus type-1 latency: current clinical realities and future scientific possibilities. Antiviral Res 48(3), 143-176.
Chaudhary, V. K., T. Mizukami, T. R. Fuerst, D. J. FitzGerald, B. Moss, I. Pastan, and E. A. Berger. 1988. Selective killing of HIV-infected cells by recombinant human CD4-Pseudomonas exotoxin hybrid protein. Nature 335:369-372.
Chun, T. W., L. Carruth, D. Finzi, X. Shen, J. A. DiGiuseppe, H. Taylor, M. Hennankova, K. Chadwick, J. Margolick, T. C. Quinn, Y. H. Kuo, R. Brookmeyer, M. A. Zeiger, P. Barditch-Crovo, and R. F. Siliciano. 1997. Quantification of latent tissue reservoirs and total body viral load in HIV-infection. Nature 387:183-188.
Chun, T. W., R. T. Davey, Jr., M. Ostrowski, J. Shawn Justement, D. Engel, J. I. Mullins, and A. S. Fauci. 2000. Relationship between pre-existing viral reservoirs and the re-emergence of plasma viremia after discontinuation of highly active anti-retroviral therapy. Nat. Med. 6:757-761.
Chun, T. W., D. Engel, M. M. Berrey, T. Shea, L. Corey, and A. S. Fauci. 1998. Early establishment of a pool of latently infected, resting CD4(+) T cells during primary HIV-1 infection. Proc. Natl. Acad. Sci. USA 95:8869-8873.
Chun, T. W., D. Engel, S. B. Mizell, C. W. Hallahan, M. Fischette, S. Park, R. T. Davey, Jr., M. Dybul, J. A. Kovacs, J. A. Metcalf, J. M. Mican, M. M. Berrey, L. Corey, H. C. Lane, and A. S. Fauci. 1999. Effect of interleukin-2 on the pool of latently infected, resting CD4+ T cells in HIV-1-infected patients receiving highly active anti-retroviral therapy. Nat Med 5:651-655.
Chun, T. W., and A. S. Fauci. 1999. Latent reservoirs of HIV: obstacles to the eradication of virus. Proc. Natl. Acad. Sci. USA 96:10958-10961.
Chun, T. W., D. Finzi, J. Margolick, K. Chadwick, D. Schwartz, and R. F. Siliciano. 1995. In vivo fate of HIV-1-infected T cells: quantitative analysis of the transition to stable latency. Nat. Med. 1:1284-1290.
Chun, T. W., L. Stuyver, S. B. Mizell, L. A. Ehler, J. A. Mican, M. Baseler, A. L. Lloyd, M. A. Nowak, and A. S. Fauci. 1997. Presence of an inducible HIV-1 latent reservoir during highly active antiretroviral therapy. Proc. Natl. Acad. Sci. USA 94:13193-13197.
Clements, J. E., T. Babas, J. L. Mankowski, K. Suryanarayana, M. Piatak, Jr., P. M. Tarwater, J. D. Lifson, and M. C. Zink. 2002. The central nervous system as a reservoir for simian immunodeficiency virus (SIV): steady-state levels of SIV DNA in brain from acute through asymptomatic infection. J. Infect. Dis. 186:905-913.
Cook, J. A., L. Albacker, A. August, and A. J. Henderson. 2003. CD28-dependent HIV-1 transcription is associated with Vav, Rac, and NF-kB activation. J. Biol. Chem. 278:35812-35818.
Cook, J. A., A. August, and A. J. Henderson. 2002. Recruitment of Phosphatidylinositol 3-Kinase to CD28 Inhibits HIV Transcription by a Tat-Dependent Mechanism. J. Immunol. 169:254-260.
Dybul, M., Hidalgo, B., Chun, T. W., Belson, M., Migueles, S. A., Justement, J. S., Herpin, B., Perry, C., Hallahan, C. W., Davey, R. T., Metcalf, J. A., Connors, M., and Fauci, A. S., 2002. Pilot study of the effects of intermittent interleukin-2 on human immunodeficiency virus (H)-specific immune responses in patients treated during recently acquired HIV infection. J Infect Dis 185(1), 61-68.
Elflein, K., M. Rodriguez-Palmero, T. Kerkau, and T. Hunig. 2003. Rapid recovery from T lymphopenia by CD28 superagonist therapy. Blood 102:1764-1770.
Elowitz, M. B., A. J. Levine, E. D. Siggia, and P. S. Swain. 2002. Stochastic gene expression in a single cell. Science 297:1183-1186.
Finzi, D., J. Blankson, J. D. Siliciano, J. B. Margolick, K. Chadwick, T. Pierson, K. Smith, J. Lisziewicz, F. Lori, C. Flexner, T. C. Quinn, R. E. Chaisson, E. Rosenberg, B. Walker, S. Gange, J. Gallant, and R. F. Siliciano. 1999. Latent infection of CD4+ T cells provides a mechanism for lifelong persistence of HIV-1, even in patients on effective combination therapy. Nat. Med. 5:512-517.
Folks, T. M., J. Justement, A. Kinter, S. Schnittman, J. Orenstein, G. Poli, and A. S. Fauci. 1988. Characterization of a promonocyte clone chronically infected with HIV and inducible by 13-phorbol-12-myristate acetate. J. Immunol. 140:1117-1122.
Golub, E. I., G. R. Li, and D. J. Volsky. 1991. Induction of dormant HIV-1 by sodium butyrate: involvement of the TATA box in the activation of the HIV-1 promoter. AIDS 5:663-668.
Hamer, D. H., S. Bocklandt, L. McHugh, T. W. Chun, P. M. Blumberg, D. M. Sigano, and V. E. Marquez. 2003. Rational design of drugs that induce human immunodeficiency virus replication. J. Virol. 77:10227-10236.
Jordan, A., Bisgrove, D., and Verdin, E., 2003. HIV reproducibly establishes a latent infection after acute infection of T cells in vitro. Embo J 22(8), 1868-1877.
Korin, Y. D., D. G. Brooks, S. Brown, A. Korotzer, and J. A. Zack. 2002. Effects of prostratin on T-cell activation and human immunodeficiency virus latency. J. Virol. 76:8118-8123.
Kulkosky, J., D. M. Culnan, J. Roman, G. Domadula, M. Schnell, M. R. Boyd, and R. J. Pomerantz. 2001. Prostratin: activation of latent HIV-1 expression suggests a potential inductive adjuvant therapy for HAART. Blood 98:3006-3015.
Kulkosky, J., G. Nunnari, M. Otero, S. Calarota, G. Domadula, H. Zhang, A. Malin, J. Sullivan, Y. Xu, J. DeSimone, T. Babinchak, J. Stem, W. Cavert, A. Haase, and R. J. Pomerantz. 2002. Intensification and stimulation therapy for human immunodeficiency virus type 1 reservoirs in infected persons receiving virally suppressive highly active antiretroviral therapy. J. Infect. Dis. 186:1403-1411.
Kutsch, O., E. N. Benveniste, G. M. Shaw, and D. N. Levy. 2002. Direct and quantitative single-cell analysis of human immunodeficiency virus type 1 reactivation from latency. J. Virol. 76:8776-8786.
Lafeuillade, A., C. Poggi, S. Chadapaud, G. Hittinger, M. Chouraqui, M. Pisapia, and E. Delbeke. 2001. Pilot study of a combination of highly active antiretroviral therapy and cytokines to induce HIV-1 remission. J. Acquir. Immune Defic. Syndr. 26:44-55.
Laughlin, M. A., S. Zeichner, D. Kolson, J. C. Alwine, T. Seshamma, R. J. Pomerantz, and F. Gonzalez-Scarano. 1993. Sodium butyrate treatment of cells latently infected with HIV-1 results in the expression of unspliced viral RNA. Virology 196:496-505.
Levy, D. N., G. M. Aldrovandi, O. Kutsch, and G. M. Shaw. 2004. Dynamics of HIV-1 recombination in its natural target cells. Proc. Nat. Acad. Sci. 101:4204-4209.
Levy, Y., C. Durier, R. Krzysiek, C. Rabian, C. Capitant, A. S. Lascaux, C. Michon, E. Oksenhendler, L. Weiss, J. A. Gastaut, C. Goujard, C. Rouzioux, J. Maral, J. F. Delfraissy, D. Emilie, and J. P. Aboulker. 2003. Effects of interleukin-2 therapy combined with highly active antiretroviral therapy on immune restoration in HIV-1 infection: a randomized controlled trial. AIDS 17:343-351.
Luhder, F., Y. Huang, K. M. Dennehy, C. Guntermann, I. Muller, E. Winkler, T. Kerkau, S. Ikemizu, S. J. Davis, T. Hanke, and T. Hunig. 2003. Topological requirements and signaling properties of T cell-activating, anti-CD28 antibody superagonists. J. Exp. Med. 197:955-966.
McHugh, L., S. Hu, B. K. Lee, K. Santora, P. E. Kennedy, E. A. Berger, I. Pastan, and D. H. Hamer. 2002. Increased affinity and stability of an anti-HIV-1 envelope immunotoxin by structure-based mutagenesis. J. Biol. Chem. 277:34383-34390.
Quivy, V., E. Adam, Y. Collette, D. Demonte, A. Chariot, C. Vanhulle, B. Berkhout, R. Castellano, Y. de Launoit, A. Burny, J. Piette, V. Bours, and C. Van Lint. 2002. Synergistic activation of human immunodeficiency virus type 1 promoter activity by NF-kB and inhibitors of deacetylases: potential perspectives for the development of therapeutic strategies. J. Virol. 76:11091-11103.
Roberts, B. D., G. Fang, and S. T. Butera. 1997. Influence of cell cycle on HIV-1 expression differs among various models of chronic infection. Arch. Virol. 142:1087-1099.
Rodriguez-Palmero, M., T. Hara, A. Thumbs, and T. Hunig. 1999. Triggering of T cell proliferation through CD28 induces GATA-3 and promotes T helper type 2 differentiation in vitro and in vivo. Eur. J. Immunol. 29:3914-3924.
Root, M. J., and D. H. Hamer. 2003. Targeting therapeutics to an exposed and conserved binding element of the HIV-1 fusion protein. Proc. Natl. Acad. Sci. USA 100:5016-5021.
Rosen, C. A., Sodroski, J. G., Campbell, K., and Haseltine, W. A., 1986. Construction of recombinant murine retroviruses that express the human T-cell leukemia virus type II and human T-cell lymphotropic virus type III trans activator genes. J Virol 57(1), 379-384.
Ross, I. L., C. M. Browne, and D. A. Hume. 1994. Transcription of individual genes in eukaryotic cells occurs randomly and infrequently. Immunol. Cell. Biol. 72:177-185.
Schmidt, J., K. Elflein, M. Stienekemeier, M. Rodriguez-Palmero, C. Schneider, K. V. Toyka, R. Gold, and T. Hunig. 2003. Treatment and prevention of experimental autoimmune neuritis with superagonistic CD28-specific monoclonal antibodies. J. Neuroimmunol. 140:143-152.
Schwartz, R. H.1992. Costimulation of T lymphocytes: the role of CD28, CTLA-4, and B7/BB1 in interleukin-2 production and immunotherapy. Cell 71:1065-1068.
Schwartz, R. H.2003. T cell anergy. Annu. Rev. Immunol. 21:305-334.
Scripture-Adams, D. D., D. G. Brooks, Y. D. Korin, and J. A. Zack. 2002. Interleukin-7 induces expression of latent human immunodeficiency virus type 1 with minimal effects on T-cell phenotype. J. Virol. 76:13077-13082.
Sodroski, J., Rosen, C., Wong-Staal, F., Salahuddin, S. Z., Popovic, M., Arya, S., Gallo, R. C., and Haseltine, W. A., 1985. Trans-acting transcriptional regulation of human T-cell leukemia virus type III long terminal repeat. Science 227(4683), 171-173
Swain, P. S., M. B. Elowitz, and E. D. Siggia. 2002. Intrinsic and extrinsic contributions to stochasticity in gene expression. Proc. Natl. Acad. Sci. USA 99:12795-12800.
Tacke, M., G. Hanke, T. Hanke, and T. Hunig. 1997. CD28-mediated induction of proliferation in resting T cells in vitro and in vivo without engagement of the T cell receptor: evidence for functionally distinct forms of CD28. Eur. J. Immunol. 27:239-247.
Tanaka, J., T. Ishida, B. I. Choi, J. Yasuda, T. Watanabe, and Y. Iwakura. 2003. Latent HIV-1 reactivation in transgenic mice requires cell cycle-dependent demethylation of CREB/ATF sites in the LTR. AIDS 17:167-175.
Tobiume, M., K. Fujinaga, M. Kameoka, T. Kimura, T. Nakaya, T. Yamada, and K. Ikuta. 1998. Dependence on host cell cycle for activation of human immunodeficiency virus type 1 gene expression from latency. J. Gen. Virol. 79:1363-1371.
Valdez, H., E. Connick, K. Y. Smith, M. M. Lederman, R. J. Bosch, R. S. Kim, M. St Clair, D. R. Kuritzkes, H. Kessler, L. Fox, M. Blanchard-Vargas, and A. Landay. 2002. Limited immune restoration after 3 years' suppression of HIV-1 replication in patients with moderately advanced disease. AIDS 16:1859-1866.
Vinay, D. S., and B. S. Kwon. 1998. Role of 4-1BB in immune responses. Semin. Immunol. 10:481-489.
Warrington, K. J., A. N. Vallejo, C. M. Weyand, and J. J. Goronzy. 2003. CD28 loss in senescent CD4+ T cells: reversal by interleukin-12 stimulation. Blood 101:3543-3549.
Willems, F., F. Andris, D. Abramowicz, G. Pierard, M. Wissing, O. Leo, and M. Goldman. 1995. Induction of T-cell anergy by OKT3 requires cyclosporine-insensitive activation signals. Transplant. Proc. 27:1425-1427.
Willems, F., F. Andris, D. Xu, D. Abramowicz, M. Wissing, M. Goldman, and O. Leo. 1995. The induction of human T cell unresponsiveness by soluble anti-CD3 mAb requires T cell activation. Int. Immunol. 7:1593-1598.

What is claimed is:

1. A cell comprising:
a) a stably integrated reporter plasmid, wherein the plasmid comprises a nucleic acid encoding a detectable reporter marker and wherein the nucleic acid is operatively linked to an immunodeficiency viral promoter; and
b) an immunodeficiency virus integrated into the genome of the cell, wherein, under basal in vitro culture conditions, the immunodeficiency virus is latent and wherein expression of the latent immunodeficiency virus can be activated.

2. The cell of claim 1, wherein the cell is an immortalized cell line.

3. The cell of claim 1, wherein the cell is a T-cell.

4. The cell of claim 1, wherein in the immunodeficiency viral promoter is a human immunodeficiency virus (HIV) promoter.

5. The cell of claim 1, wherein the immunodeficiency virus is human immunodeficiency virus (HIV).

6. The cell of claim 5, wherein the immunodeficiency virus is HIV-1.

7. The cell of claim 1, wherein the detectable reporter marker is enhanced green fluorescence protein (EGFP).

8. The cell of claim 1, wherein the immunodeficiency virus is activated.

9. A method of identifying an agent that activates a latent immunodeficiency virus, the method comprising:
a) contacting the cell of claim 1 with a test agent; and
b) detecting the detectable reporter marker, the detectable reporter marker indicating that the test agent activates the latent immunodeficiency virus.

10. A composition comprising:
a) an agent identified by the method of claim 9; and
b) a pharmaceutically acceptable carrier.

11. A method of treating a subject with human immunodeficiency virus comprising:
a) administering to the subject an effective amount of the composition of claim 10; and
b) administering to the subject an effective amount of one or more agents that inhibit a human immunodeficiency viral function.

12. The method of claim 11, wherein the agent or agents that inhibit the human immunodeficiency virus function are selected from the group consisting of a viral replication inhibitor, a viral protease inhibitor, a viral reverse transcriptase inhibitor, a viral entry inhibitor, a viral integrase inhibitor, a viral Rev inhibitor, a viral Tat inhibitor, a viral Nef inhibitor, a viral Vpr inhibitor, a viral Vpu inhibitor, and a viral Vif inhibitor.

13. A method of identifying an agent that causes activation of a target cell, the method comprising:
a) contacting the cell of claim 1 with a test agent; and
b) detecting the detectable reporter marker, the detectable reporter marker indicating that the test agent activates the target cell.

14. The method of claim 13, wherein the target cell is a T-cell.

15. A composition comprising, an agent identified by the method of claim 13 and a pharmaceutically acceptable carrier.

16. A method of activating a latent pathogen in a cell, comprising contacting the cell with an effective amount of an agent identified by the method of claim 13.

17. The method of claim 16, wherein the latent pathogen is selected from the group consisting of a herpes virus, a hepatitis virus, a mycobacterium, a mycoplasma, and a toxoplasma.

18. A method of enhancing an immune response in a subject comprising administering to the subject an effective amount of the composition of claim 15.

19. A vaccine adjuvant, comprising an agent identified by the method of claim 13.

20. A method of identifying an agent that inhibits immunodeficiency virus transcription, comprising:
a) contacting the cell of claim 1 with a test agent;
b) contacting the cell of claim 1 with an immunodeficiency virus activating agent; and
c) detecting the presence of detectable reporter marker, the level of which, when decreased as compared to a control level, indicating an agent that inhibits viral transcription.

21. The method of claim 20, wherein the immunodeficiency virus activating agent activates HIV-1.

22. A composition comprising an agent identified by the method of claim 20 and a pharmaceutically acceptable carrier.

23. A method of inhibiting viral transcription in a cell, comprising contacting the cell with an agent identified by the method of claim 20.

24. A method of treating a subject with human immunodeficiency virus infection, comprising administering to the subject an effective amount of the composition of claim 22.

25. The method of claim 24 further comprising:
a) administering to the subject an effective amount of the composition of claim 22; and
b) administering to the subject an effective amount of one or more agents that inhibit a human immunodeficiency viral function.

26. The method of claim 25, wherein the agent or agents that inhibit the human immunodeficiency virus function are selected from the group consisting of a viral replication inhibitor, a viral protease inhibitor, a viral reverse transcriptase inhibitor, a viral entry inhibitor, a viral integrase inhibitor, a viral Rev inhibitor, a viral Tat inhibitor, a viral Nef inhibitor, a viral Vpr inhibitor, a viral Vpu inhibitor, and a viral Vif inhibitor.

27. A method of activating a latent immunodeficiency virus in a subject, comprising administering to the subject an effective amount of activating anti-CD28 antibody.

28. The method of claim 27, further comprising:
a) administering to the subject an effective amount of activating anti-CD28 antibody; and
b) administering to the subject an effective amount of one or more agents that inhibit a human immunodeficiency viral function.

29. The method of claim 28, wherein the agent or agents that inhibit the human immunodeficiency virus function are selected from the group consisting of a viral replication inhibitor, a viral protease inhibitor, a viral reverse transcriptase inhibitor, a viral entry inhibitor, a viral integrase inhibitor, a viral Rev inhibitor, a viral Tat inhibitor, a viral Nef inhibitor, a viral Vpr inhibitor, a viral Vpu inhibitor, and a viral Vif inhibitor.

30. A method of treating a subject infected with HIV, comprising:
a) extracting bone marrow stem cells or peripheral blood stem cells from the subject;
b) eradicating all T cells in the subject;
c) contacting the extracted stem cells with an effective amount of an agent identified using the method of claim 9, wherein contacting the bone marrow with the agent activates latent HIV expression in the extracted cell population;
d) contacting the extracted stem cells with an effective amount of one or more agents that kill stem cells with active HIV expression; and
e) transplanting the surviving stem cells into the subject.

31. The cell of claim 1, further comprising:
a stably integrated indicator plasmid, wherein the indicator plasmid comprises a nucleic acid encoding a detectable indicator marker distinguishable from the detectable reporter marker, wherein the detectable indicator marker is constitutively expressed under basal in vitro culture conditions.

32. The cell of claim 31, wherein the detectable reporter marker and the detectable indicator marker are fluorescent proteins spectrally distinguishable from each other.

33. The cell of claim 31, wherein the detectable reporter marker is enhanced green fluorescence protein (EGFP) and wherein the detectable indicator marker is a fluorescent protein spectrally distinguishable from EGFP.

34. The cell of claim 31, wherein the detectable indicator marker is a detectable protein.

35. The cell of claim 32, wherein the detectable protein is selected from the group consisting of luciferase, soluble alkaline phosphatase, and a cell surface-expressed marker protein.

36. A method of identifying an agent that activates a latent immunodeficiency virus, the method comprising:
a) contacting the cell of claim 31 with a test agent; and
b) detecting the detectable reporter marker, the detectable reporter marker indicating that the test agent activates the latent immunodeficiency virus.

37. The method of claim 36, further comprising detecting the detectable indicator marker, a stable level of the indicator marker as compared to a control level indicating that the test agent is not cytotoxic to the cell.

38. The method of claim 36, further comprising detecting the detectable indicator marker, a decrease in the level of the detectable indicator marker as compared to a control level indicating cytotoxicity of the test agent.

39. The method of claim 38, wherein the death of the cell indicates a cytotoxic test agent.

40. A composition comprising:
a) an agent identified by the method of claim 36; and
b) a pharmaceutically acceptable carrier.

41. A method of treating a subject with human immunodeficiency virus, comprising:
a) administering to the subject an effective amount of the composition of claim 40; and
b) administering to the subject an effective amount of one or more agents that inhibit a human immunodeficiency viral function.

42. The method of claim 41, wherein the agent or agents that inhibit the human immunodeficiency virus function are selected from the group consisting of a viral replication inhibitor, a viral protease inhibitor, a viral reverse transcriptase inhibitor, a viral entry inhibitor, a viral integrase inhibitor, a viral Rev inhibitor, a viral Tat inhibitor, a viral Nef inhibitor, a viral Vpr inhibitor, a viral Vpu inhibitor, and a viral Vif inhibitor.

43. A method of identifying an agent that causes activation of a target cell, the method comprising:
a) contacting the cell of claim 31 with a test agent; and
b) detecting the detectable reporter marker, the detectable reporter marker indicating that the test agent activates the target cell.

44. The method of claim 43, further comprising detecting the detectable indicator marker, a stable level of the indicator marker as compared to a control level indicating that the test agent is not cytotoxic to the cell.

45. The method of claim 43, further comprising detecting the detectable indicator marker, a decrease in the level of the detectable indicator marker as compared to a control level indicating cytotoxicity of the test agent.

46. The method of claim 43, wherein the target cell is a T-cell.

47. A composition comprising, an agent identified by the method of claim 43 and a pharmaceutically acceptable carrier.

48. A method of activating a latent pathogen in a cell, comprising contacting the cell with an effective amount of an agent identified by the method of claim 43.

49. The method of claim 48, wherein the latent pathogen is selected from the group consisting of a herpes virus, a hepatitis virus, a mycobacterium, a mycoplasma, and a toxoplasma.

50. A method of enhancing an immune response in a subject, comprising administering to the subject an effective amount of the composition of claim 47.

51. A vaccine adjuvant comprising an agent identified by the method of claim 43.

52. A method of identifying an agent that inhibits immunodeficiency virus transcription, comprising:
a) contacting the cell of claim 31 with a test agent and an immunodeficiency virus activating agent; and
b) detecting the presence of the detectable reporter marker, a decreased level of the detectable reporter marker as compared to a control level, indicating an agent that inhibits viral transcription.

53. The method of claim 52, further comprising detecting the detectable indicator marker, a stable level of the indicator marker as compared to a control level indicating that the test agent is not cytotoxic to the cell.

54. The method of claim 52, further comprising detecting the detectable indicator marker, a decrease in the level of the detectable indicator marker as compared to a control level indicating cytotoxicity of the test agent.

55. The method of claim 52, wherein the immunodeficiency virus activating agent activates HIV-1.

56. A composition, comprising an agent identified by the method of claim 52 and a pharmaceutically acceptable carrier.

57. A method of inhibiting viral transcription in a cell, comprising contacting the cell with an agent identified by the method of claim 52.

58. A method of treating a subject with human immunodeficiency virus infection, comprising administering to the subject an effective amount of the composition of claim 56.

59. The method of claim 58, further comprising:
a) administering to the subject an effective amount of the composition of claim 56; and
b) administering to the subject an effective amount of one or more agents that inhibit a human immunodeficiency viral function.

60. The method of claim 59, wherein the agent or agents that inhibit the human immunodeficiency virus function are selected from the group consisting of a viral replication inhibitor, a viral protease inhibitor, a viral reverse transcriptase inhibitor, a viral entry inhibitor, a viral integrase inhibitor, a viral Rev inhibitor, a viral Tat inhibitor, a viral Nef inhibitor, a viral Vpr inhibitor, a viral Vpu inhibitor, and a viral Vif inhibitor.

61. A method of treating a subject infected with HIV, comprising:
a) extracting bone marrow stem cells or peripheral blood stem cells from the subject;
b) eradicating all T cells in the subject;
c) contacting the extracted stem cells with an effective amount of an agent identified using the method of claim 31, wherein contacting the bone marrow with the agent activates latent HIV expression in the extracted cell population;
d) contacting the extracted stem cells with an effective amount of one or more agents that kill stem cells with active HIV expression; and
e) transplanting the surviving stem cells into the subject.


SEQUENCE LISTING (1 sequence)

SEQ ID NO: 1 - DNA, 555 bp, Artificial Sequence
  Description of Artificial Sequence, note = synthetic
gactggaagggctaattcactcccaacgaagacaagatatccttgatctgtggatctaccacacacaaggctacttccctgattggcagaactacacaccagggccaggagtcagatatccactgacctttggatggtgctacaagctagtaccagttgagccagataaggtagaagaggccaacaaaggagagaacaccagcttgttacaccctgtgagcctgcatggaatggatgacccggagagagaagtgttagagtggaggtttgacagccgcctagcatttcatcacatggcccgagagctgcatccggagtacttcaagaactgctgacatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactaaggaacccactgcttaagcctcaataaagcttgccttgagtgcttcaa